(19) United States
(12) Patent Application Publication
(10) Pub. No.: US 2016/0235810 A1
(43) Pub. Date: Aug. 18, 2016

(54) METHODS OF TREATING DIABETES AND RELATED DISORDERS

(71) Applicants: Avirup BOSE, Cambridge, MA (US); Alokesh DUTTAROY, Cambridge, MA (US)

(72) Inventors: Avirup BOSE, Marlborough, MA (US); Alokesh DUTTAROY, Marlborough, MA (US)

(73) Assignee: NOVARTIS AG, Basel (CH)

(21) Appl. No.: 15/029,689

(22) PCT Filed: Oct. 16, 2014
(86) PCT No.: PCT/US2014/060807

§ 371 Date: Apr. 15, 2016

Related U.S. Application Data
(60) Provisional application No. 61/892,550, filed on Oct. 18, 2013.

(51) Int. Cl. A61K 38/17

(52) U.S. Cl. CPC A61K 38/1709

(57) ABSTRACT

The invention relates to methods of treating metabolic conditions, including type 2 diabetes mellitus, by administering prolactin-inducible protein (PIP) or fusion proteins, analogs or variants of PIP with altered or truncated PIP sequence, site-specific mutants, or chemically modified mutants including pharmaceutical properties comprising the same.

### FIELD OF THE INVENTION

The present invention relates to new methods for treating metabolic disorders such as type 2 diabetes mellitus (T2DM), pancreatic beta cell impairment, glucose intolerance, hyperglycemia, insulin resistance, obesity, dyslipidemia, nonalcoholic steatohepatitis (NASH), metabolic syndrome, and other metabolic disorders.

### BACKGROUND OF THE INVENTION

Prolactin-inducible protein (PIP), also known as gross cystic disease fluid protein (GCDFP-15), is expressed in exocrine organs, and in benign and malignant human breast tumors.

The mature secreted PIP protein has a molecular mass of 13 kDa and it runs as a 17 kDa polypeptide in SDS-PAGE. PIP is expressed in most organs that contribute to human body fluids. The expression of PIP is highest in salivary gland followed by lacrimal, prostrate, muscle, trachea and mammary glands. Although the exact functions of PIP are not known, several possible functions including inhibition of bacterial growth and immune regulation have been proposed. Reports suggest that PIP can be used as a biomarker for tumor progression in breast cancer.

### SUMMARY OF THE INVENTION

The invention relates to the present discovery that PIP can enhance insulin secretion, enhance insulin sensitivity and reduce plasma glucose concentration, as described further, and as experimentally shown, herein. Although earlier reports suggested that PIP plays biological roles in antimicrobial activity, immunoregulation, fertility, and tumor progression, this is the first instance linking it to regulation of pancreatic function, insulin sensitivity and glucose control for the treatment of T2DM.

The proposed invention is a PIP conjugated with Fc or human serum albumin (HSA) or His-tag or proprietary APP tags. Other proprietary PIP variants with altered or truncated PIP sequences, site-specific mutations, or chemically modified mutants to extend the stability, half-life and improved therapeutic profile of PIP are also claimed.

The present invention is drawn to methods of treating a patient exhibiting one or more metabolic disorders such as T2DM, pancreatic beta cell impairment, glucose intolerance, hyperglycemia, insulin resistance, obesity, dyslipidemia, NASH, metabolic syndrome and other metabolic disorders, comprising administering to said patient in need of such treatment a therapeutically effective amount of one or more human PIP polypeptides, analogs, fusion proteins, or pharmaceutical compositions thereof.

The present invention is drawn to methods of improving pancreatic beta cell function in subjects in need, comprising administering to said subject a therapeutically effective amount of one or more human PIP polypeptides, analogs, fusion proteins, or pharmaceutical compositions thereof.

The present invention is drawn to methods of improving glucose control in subjects in need, comprising administering to said subject a therapeutically effective amount of one or more human PIP polypeptides, analogs, fusion proteins, or pharmaceutical compositions thereof.

The present invention is drawn to methods of improving insulin sensitivity in subjects in need, comprising administering to said subject a therapeutically effective amount of one or more human PIP polypeptides, analogs, fusion proteins, or pharmaceutical compositions thereof.

The present invention is drawn to methods for reducing one or more metabolic disorders such as hyperglycemia, excess liver lipids, and weight gain in subjects in need, comprising administering to said subject a therapeutically effective amount of one or more human PIP polypeptides, analogs, fusion proteins, or pharmaceutical compositions thereof.

The methods of the present invention include native (wild type) PIP and PIP analogs including variants, truncated versions, fusions with Fc, albumin, polyethylene glycol (PEG) and with other peptides, proteins or lipids, including but not limited to GLP-1 and Exendin-4, that have glucose lowering effects. The methods of the present invention also include anti-PIP antibodies that increase its plasma half-life and/or enhance (agonize) its biological activity, and anti-PIP receptor (PIPR) antibodies that activate PIP signaling.

The methods of the present invention include novel PIP analogs including variants and truncated versions, as well as fusions of said analogs with Fc, albumin, PEG and with other peptides, proteins or lipids, including but not limited to GLP-1 and Exendin-4, that have glucose lowering effects. The present invention also includes novel anti-PIP antibodies that increase plasma half-life of PIP and/or enhance (agonize) its biological activity, and novel anti-PIPR antibodies that activate PIP signaling.

As used herein, the terms “protein variant”, “human variant”, “polypeptide or protein variant”, “variant”, “mutant”, as well as any like terms or specific versions thereof (e.g., “PIP variant”, “human GLP-1 variant”, “Fibroblast Growth factor (FGF21) mutant”, etc.) define protein or polypeptide sequences that comprise modifications, truncations or other variants of naturally occurring (i.e., wild-type) protein or polypeptide counterparts or corresponding native sequences. “Variant PIP” or “PIP mutant,” for instance, is described relative to the wild-type (i.e., naturally occurring) PIP as described herein and known in the literature.

Similarly, as used herein, the term “compositions of the present invention”, as well as any like terms, when applying to PIP, define proteins, peptides, polypeptides, analogs, variants, and fusion proteins and pharmaceutical compositions thereof embodied by the present methods and compositions.

These and other aspects of the invention will be elucidated in the following detailed description of the invention.

### BRIEF DESCRIPTION OF THE DRAWINGS

FIGS. 1 A and B are a graphical representations of body weight (A) and food intake (B) time course among Sham-Ad libitum (as shown by triangles), Sham-pair fed (as shown by diamonds), and Roen-en-Y gastric bypass (RYGB) groups (as shown by circles). **p<0.01, ***p<0.001 by two-way ANOVAs with post hoc t-tests for each time point.

FIGS. 2 A and B are a graphical representations of pancreatic beta cell function as quantified by the insulin area under the curves above background (AUCB) during either an glucose+arginine challenge (A, in Zucker Diabetic Fatty (ZDF) rats) or an oral glucose challenge (B, in Goto-Kakizaki (GK) rats) for RYGB and Sham-pair fed (Sham-PF) groups. **p<0.01 by two-way ANOVAs with post hoc t-tests for the excursion and AUCB.

FIGS. 3 A and B are a graphical representations of pancreatic beta cell immunohistochemistry (3A:ZDF; 3B:GK). Right panels show a representative islet staining picture and left panels show the quantified % islet fibrotic area (non-beta cell stained area). RYGB surgery reduced the % fibrotic area compared to Sham-PF groups. **p<0.01 by t-tests.

FIGS. 4 A and B are graphical representations showing that the administration of mouse PIP via hydrodynamic gene delivery in high fat diet-induced obese (DIO) mice increased glucose-stimulated insulin secretion (GSIS) and reduced glucose levels. There was a 72% increase in insulin area under the curve (4A) and a 30% reduction in glucose area under the curve (4B) compared to the vector control group. *p<0.05, **p<0.01 by t-test.

FIGS. 5 A and B are graphical representations showing that a single intraperitoneal administration of a human serum albumin-recombinant human PIP fusion protein (at 0.3 mg/kg, IP) increased GSIS and improved oral glucose tolerance (OGT) in high fat DIO mice at 1 h post-dose. There was a 60% increase in insulin area under the curve (GSIS) (5A) and a 36% reduction in glucose area under the curve (5B) compared to the Vehicle control group; *p<0.05 by t-test.

FIG. 6 is a graphical representation showing that a HSA-recombinant human PIP fusion protein (0.3 mg/kg, IP) was additive to Exendin-4 (0.3 ug/kg, IP) to improve oral glucose tolerance (OGT; dextrose 3 g/kg, oral given at 1 h post-dose) in high fat DIO mice. Data are presented as a time-course of glucose excursion *p<0.05, **p<0.01 from Control, +p<0.05 from Exendin-4 by a 2-way ANOVA with post-hoc analysis.

FIG. 7 is a graphical representation showing that the Fc-recombinant human PIP fusion protein (0.1 m/kg, IP) was additive to Metformin (100 mg/kg, oral) to improve insulin sensitivity as measured by an insulin tolerance test (ITT, Humulin 1.2 U/kg, IP was given at 1 h post-dose) in the insulin resistant ob/ob mice. Data are presented as a time-course of glucose excursion. *p<0.05 from Control, +p<0.05 from Metformin by a 2-way ANOVA with post-hoc analysis.

### DETAILED DESCRIPTION OF THE INVENTION

Expression of the mouse paralog of PIP, Seminal vesicle antigen-like 1 (Sval1), was increased in the ileum following Roux-en-Y gastric bypass (RYGB) surgery, as described below. Additional experiments, in which Sval1, and PIP, as described below, were overexpressed from the pLEV113 vector via hydrodynamic injection, showed an improvement in GSIS with corresponding plasma glucose reduction in DIO mice.

Genetic and environmental factors have been widely implicated in the development of insulin resistance in humans. If left untreated, insulin resistance can lead to beta cell failure and hyperglycemia, a hallmark of T2DM. Therefore, improvement of beta cell function improves glycemic control in patients with T2DM. However, the currently available insulin secretagogues approved for therapeutic purposes have several undesirable effects including hypoglycemia, nausea and pancreatitis, which highlights the importance of identifying new agents that can improve beta cell function with a more desirable profile.

It has been shown that gastric bypass surgery, such as Roux-en-Y Gastric Bypass (RYGB) surgery, cures diabetes in a vast majority of obese diabetic patients that undergo the surgical procedure. In many cases, the patients become normoglycemic before any decline in body weight. It is widely believed that improvement in insulin sensitivity precedes loss in body weight and is primarily responsible for improving glycemic control in the early days after surgery. We therefore initiated studies to identify the factor(s) that are responsible for these post-surgical metabolic improvements. This led to the methods of the invention and revealed that expression of the paralog of the human PIP gene, Sval1, is upregulated after gastric bypass surgery and revealed PIP as capable of improving GSIS, insulin sensitivity and glycemic control in vivo.

The present invention is drawn to methods of treating a patient exhibiting one or more metabolic disorders such as T2DM, glucose intolerance, pancreatic beta cell impairment, hyperglycemia, insulin resistance, obesity, dyslipidemia, NASH, metabolic syndrome and other metabolic disorders, comprising administering to said patient in need of such treatment a therapeutically effective amount of one or more human PIP polypeptides, analogs, variants or fusion proteins, or pharmaceutical compositions thereof.

The present invention is drawn to novel compositions of matter that can be used to treat a patient exhibiting one or more metabolic disorders such as T2DM, glucose intolerance, pancreatic beta cell impairment, hyperglycemia, insulin resistance, obesity, dyslipidemia, NASH, metabolic syndrome and other metabolic disorders, and in reducing the mortality and morbidity of critically ill patients, said compositions comprising one or more human PIP polypeptides, analogs, variants or fusion proteins, or pharmaceutical compositions thereof.

### DEFINITIONS

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art.

As used herein, the term “PIP” refers to prolactin-inducible protein, a protein with GenBank Accession No. NP_002643 that exhibits roles in diverse biological processes. PIP is also known in the art as gross cystic disease fluid protein-15 (GCDFP-15); secretory actin-binding protein (SABP); extraparotid glycoprotein (EP-GP); and 17-kDa CD4-binding protein (GP17).

The term “isolated nucleic acid molecule” refers to a nucleic acid molecule of the present invention that (1) has been separated from at least about 50 percent of proteins, lipids, carbohydrates, or other materials with which it is naturally found when total nucleic acid is isolated from the source cells, (2) is not linked to all or to a portion of a polynucleotide to which the “isolated nucleic acid molecule” is linked in nature, or (3) is operably linked to a polynucleotide which it is not linked to in nature. Preferably, the isolated nucleic acid molecule of the present invention is substantially free from any other contaminating nucleic acid molecules or other contaminants that are found in its natural environment that would interfere with its use in polypeptide production or its therapeutic, diagnostic, prophylactic or research use.

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “amino acid,” as used herein, refers to naturally occurring amino acids, unnatural amino acids, amino acid analogues and amino acid mimetics that function in a manner similar to the naturally occurring amino acids, all in their D and L stereoisomers if their structure allows such stereoisomeric forms. Amino acids are referred to herein by either their name, their commonly known three letter symbols or by the one-letter symbols recommended by the IUPAC-IUB Biochemical Nomenclature Commission.

The term “naturally occurring” when used in connection with biological materials such as nucleic acid molecules, polypeptides, host cells, and the like, refers to materials that are found in nature and are not manipulated by man. Similarly, “non-naturally occurring” as used herein refers to a material that is not found in nature or that has been structurally modified or synthesized by man. When used in connection with nucleotides, the term “naturally occurring” refers to the bases adenine (A), cytosine (C), guanine (G), thymine (T), and uracil (U). When used in connection with amino acids, the term “naturally occurring” refers to the 20 conventional amino acids (i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y)), as well as selenocysteine, pyrrolysine (PYL), and pyrroline-carboxy-lysine (PCL).

The term “amino acid mimetics,” as used herein, refers to chemical compounds that have a structure that is different from the general chemical structure of an amino acid, but functions in a manner similar to a naturally occurring amino acid.

The term “biologically active variant” refers to any PIP polypeptide analog or variant used in the invention that possesses an activity of wild-type (e.g., naturally-occurring) PIP, such as the ability to improve insulin secretion, insulin sensitivity and/or glycemic control. Polypeptide variants possessing a somewhat decreased level of activity relative to their wild-type versions can nonetheless be considered to be biologically active polypeptide variants, although ideally a biologically active polypeptide possesses similar or enhanced biological properties relative to its wild-type protein counterpart.

The terms “effective amount” and “therapeutically effective amount” each refer to the amount of a PIP, fusion protein or analog used to support an observable level of one or more biological activities of wild-type PIP, such as the ability to lower blood glucose, increase glucose or nutrient-stimulated insulin secretion, lower triglyceride levels, reduce liver triglyceride or lipid levels, induce weight loss, or improve glucose tolerance or insulin sensitivity. For example, a “therapeutically-effective amount” administered to a patient exhibiting, suffering, or prone to suffer from metabolic disorders (such as T2DM, obesity, or metabolic syndrome), is such an amount which induces, ameliorates or otherwise causes an improvement in the pathological symptoms, disease progression, physiological conditions associated with or resistance to succumbing to the aforementioned disorders. For the purposes of the present invention a “subject” or “patient” is preferably a human, but can also be an animal, more specifically, a companion animal (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “physiologically acceptable carrier” as used herein refers to one or more formulation materials suitable for accomplishing or enhancing the delivery of PIP, an analog, or fusion protein.

The term “antigen” refers to a molecule or a portion of a molecule that is capable of being bound by an antibody, and additionally that is capable of being used in an animal to produce antibodies that are capable of binding to an epitope of that antigen. An antigen may have one or more epitopes.

The term “native Fc” refers to a molecule or sequence comprising the sequence of a non-antigen-binding fragment resulting from digestion of whole antibody or produced by other means, whether in monomeric or multimeric form, and can contain the hinge region. The original immunoglobulin source of the native Fc is preferably of human origin and can be from any of the immunoglobulins, although IgG1 and IgG2 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG (see Ellison et al., (1982), Nucleic Acids Res. 10: 4071-9). The term “native Fc” as used herein is generic to the monomeric, dimeric, and multimeric forms. The term “Fc variant” refers to a molecule or sequence that is modified from a native Fc but still comprises a binding site for the salvage receptor, FcRn (neonatal Fc receptor). International Publication Nos. WO 97/34631 and WO 96/32478 describe exemplary Fc variants, as well as interaction with the salvage receptor, and are hereby incorporated by reference. Thus, the term “Fc variant” can comprise a molecule or sequence that is humanized from a non-human native Fc. Furthermore, a native Fc comprises regions that can be removed because they provide structural features or biological activity that are not required for the fusion molecules of PIP or analogs of the present methods and compositions of the invention.

Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variants and sequences as defined above. As with Fc variants and native Fc molecules, the term “Fc domain” includes molecules in monomeric or multimeric form, whether digested from whole antibody or produced by other means. In some embodiments of the present invention, an Fc domain can be fused to a PIP (including a truncated form of PIP) or analog via, for example, a covalent bond between the Fc domain and the PIP or analog sequence. Such fusion proteins can form multimers via the association of the Fc domains and both these fusion proteins and their multimers are an aspect of the present invention.

The term “polyethylene glycol” or “PEG” refers to a polyalkylene glycol compound or a derivative thereof, with or without coupling agents or derviatization with coupling or activating moieties.

The term “metabolic disorders,” and terms similarly used herein, includes but is not limited to obesity, T2DM, pancreatitis, dyslipidemia, NASH, insulin resistance, hyperinsulinemia, glucose intolerance, hyperglycemia, metabolic syndrome, hypertension, cardiovascular disease, atherosclerosis, peripheral arterial disease, stroke, heart failure, coronary heart disease, kidney disease, diabetic complications, neuropathy, gastroparesis and other metabolic disorders.

“Type 2 diabetes mellitus (T2DM)” is a condition characterized by excess blood glucose concentration in spite of the availability of insulin.

“Dyslipidemia” is a disorder of lipoprotein metabolism, including lipoprotein overproduction or deficiency. Dyslipidemias may be manifested by elevation of the total cholesterol, low-density lipoprotein (LDL) cholesterol or triglyceride concentrations, or a decrease in high-density lipoprotein (HDL) cholesterol concentration in the blood.

“Nonalcoholic steatohepatitis (NASH)” is a liver disease, not associated with alcohol consumption, characterized by fatty change of hepatocytes, accompanied by intralobular inflammation and fibrosis.

“Glucose intolerance” or “Impaired Glucose Tolerance” (IGT) is a pre-diabetic state of dysglycemia that is associated with increased risk of cardiovascular pathology. The pre-diabetic condition prevents a subject from moving glucose into cells efficiently and utilizing it as an efficient fuel source, leading to elevated glucose levels in blood and some degree of insulin resistance.

“Hyperglycemia” is defined as an excess of sugar (glucose) in the blood.

“Hypoglycemia,” also called low blood sugar, occurs when blood glucose level is too low to provide enough energy for the body's activities.

“Hyperinsulinemia” is defined as a higher-than-normal level of insulin in the blood.

“Insulin resistance” is defined as a state in which a normal amount of insulin produces a subnormal biologic response.

“Obesity,” in terms of the human subject, can be defined as an adult with a Body Mass Index (BMI) of 30 or greater (Centers for Disease Control and Prevention).

“Metabolic syndrome” can be defined as a cluster of at least three of the following signs: abdominal fat—in most men, a 40-inch waist or greater; high blood glucose—at least 110 milligrams per deciliter (mg/dl) after fasting; high triglycerides—at least 150 mg/dL in the bloodstream; low HDL—less than 40 mg/dl; and, blood pressure of 130/85 mmHg or higher.

“Hypertension” or high blood pressure that is a transitory or sustained elevation of systemic arterial blood pressure to a level likely to induce cardiovascular damage or other adverse consequences. Hypertension has been arbitrarily defined as a systolic blood pressure above 140 mmHg or a diastolic blood pressure above 90 mmHg.

“Cardiovascular diseases” are diseases related to the heart or blood vessels.

“Atherosclerosis” is a vascular disease characterized by irregularly distributed lipid deposits in the intima of large and medium-sized arteries, sometimes causing narrowing of arterial lumens and proceeding eventually to fibrosis and calcification. Lesions are usually focal and progress slowly and intermittently. Limitation of blood flow accounts for most clinical manifestations, which vary with the distribution and severity of lesions.

“Stroke” is any acute clinical event, related to impairment of cerebral circulation, that lasts longer than 24 hours. A stroke involves irreversible brain damage, the type and severity of symptoms depending on the location and extent of brain tissue whose circulation has been compromised.

“Heart failure”, also called congestive heart failure, is a condition in which the heart can no longer pump enough blood to the rest of the body.

“Coronary heart disease”, also called coronary artery disease, is a narrowing of the small blood vessels that supply blood and oxygen to the heart.

“Kidney disease” or nephropathy is any disease of the kidney. Diabetic nephropathy is a major cause of morbidity and mortality in people with T2DM.

“Diabetic complications” are problems caused by high blood glucose levels, with body functions such as kidneys, nerves (neuropathies), feet (foot ulcers and poor circulation) and eyes (e.g. retinopathies). Diabetes also increases the risk for heart disease and bone and joint disorders. Other long-term complications of diabetes include skin problems, digestive problems, sexual dysfunction and problems with teeth and gums.

“Neuroapathies” are any diseases involving the cranial nerves or the peripheral or autonomic nervous system.

“Gastroparesis” is weakness of gastric peristalsis, which results in delayed emptying of the bowels.

As used herein, the singular forms “a,” “an” and “the” include plural references unless the content clearly dictates otherwise. Thus, for example, reference to “an antibody” includes a mixture of two or more such antibodies.

As used herein, the term “about” refers to +/−20%, +/−10%, or +/−5% of a value.

The terms “polypeptide” and “protein”, are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous signal sequences, with or without N-terminal methionine residues; immunologically tagged proteins; and the like.

The terms “individual,” “subject,” “host,” and “patient,” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, the term “sample” refers to biological material from a patient. The sample assayed by the present invention is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

As used herein, the term “biological molecule” includes, but is not limited to, peptides, polypeptides, nucleic acids, saccharides and polysaccharides.

As used herein, the term “modulating” refers to a change in the quality or quantity of a gene, protein, or any molecule that is inside, outside, or on the surface of a cell. The change can be an increase or decrease in expression or level of the molecule. The term “modulates” also includes changing the quality or quantity of a biological function/activity including, without limitation, the ability to lower blood glucose, insulin, triglyceride, or cholesterol levels; to reduce liver lipid or liver triglyceride levels; to reduce body weight; and to improve glucose tolerance, energy expenditure, or insulin sensitivity.

As used herein, the term “modulator” refers to a composition that modulates one or more physiological or biochemical processes associated with a metabolic disorder, such as T2DM or a metabolic condition such as obesity. Said events include but are not limited to the ability to lower blood glucose, insulin, triglyceride, or cholesterol levels; to reduce liver lipid or liver triglyceride levels; to reduce body weight; and to improve glucose tolerance, energy expenditure, or insulin sensitivity.

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide etc. Also encompassed by this term are biopolymeric products that are made using an RNA gene product as a template (i.e., cDNA of the RNA). A gene product may be made enzymatically, recombinantly, chemically, or within a cell to which the gene is native. In some embodiments, such as if the gene product is proteinaceous, it exhibits a biological activity. In some embodiments, if the gene product is a nucleic acid, it can be translated into a proteinaceous gene product that exhibits a biological activity.

“Modulation of PIP activity,” as used herein, refers to an increase or decrease in PIP activity that can be a result of, for example, interaction of an agent with a PIP-encoding polynucleotide, PIP polypeptide, PIP mRNA, inhibition of PIP transcription and/or translation (e.g., through antisense or siRNA interaction with the PIP gene or PIP transcript, through modulation of transcription factors that facilitate PIP expression), and the like. For example, modulation of a biological activity refers to an increase or a decrease in a biological activity. Modulation of PIP activity can be achieved by administering PIP or PIP analogs, including but not limited to wild type PIP and nucleic acids and variants and analogs thereof. Modulation of PIP activity can also be achieved by administering therapeutic mRNAs designed to deliver and increase circulating PIP levels.

PIP activity can be assessed by means including, without limitation, assaying blood glucose, insulin, triglyceride, or cholesterol levels in a subject, assessing PIP polypeptide levels, or by assessing PIP transcription levels. Comparisons of PIP activity can also be accomplished by, e.g., measuring levels of a PIP downstream biomarker, and measuring changes in PIP signaling.

PIP activity can also be assessed by measuring: cell signaling; kinase activity; glucose uptake into adipocytes or muscle cells; glucose flux into or out of hepatocytes; blood insulin, triglyceride, or cholesterol level fluctuations; liver lipid or liver triglyceride level changes; interactions between PIP and a PIP receptor; or modification of a PIP receptor, for example, by phosphorylation. In some embodiments phosphorylation of a PIP receptor can be tyrosine phosphorylation. In some embodiments modulation of PIP activity can cause modulation of a PIP-related process or phenotype.

A “PIP downstream biomarker,” as used herein, is a gene or gene product, or measurable indicia of a gene or gene product. In some embodiments, a gene or activity that is a downstream marker of PIP exhibits an altered level of expression. In some embodiments, an activity of the downstream marker is altered in the presence of a PIP modulator. In some embodiments, the downstream marker exhibits altered levels of expression when PIP is perturbed with a PIP modulator of the present invention.

As used herein, the term “up-regulates” refers to an increase, activation or stimulation of an activity or quantity. For example, in the context of the present invention, PIP modulators may increase the activity of a PIP. In one embodiment, PIP or its receptor may be upregulated in response to a PIP modulator. Upregulation can also refer to a PIP-related activity, such as e.g., the ability to lower blood glucose, insulin, triglyceride, or cholesterol levels; to reduce liver lipid or triglyceride levels; to reduce body weight; to improve glucose tolerance, energy expenditure, or insulin sensitivity; or to cause a modification (for example, phosphorylation) of a PIP receptor; or to increase a PIP downstream biomarker. Up-regulation may be at least 25%, at least 50%, at least 75%, at least 100%, at least 150%, at least 200%, at least 250%, at least 400%, or at least 500% as compared to a control.

As used herein, the term “N-terminus” refers to at least the first 10 amino acids of a protein. As used herein, the term “C-terminus” refers to at least the last 10 amino acids of a protein.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

As used herein, the term “signal domain” (also called “signal sequence” or “signal peptide”) refers to a peptide domain that resides in a continuous stretch of amino acid sequence, often at the N-terminal region of a precursor protein (often a membrane-bound or secreted protein), and is involved in protein transport to a particular cellular destination, for example the endoplasmic reticulum or mitochondria. In many cases the signal domain is removed from the full-length protein by specialized signal peptidases after the sorting process has been completed. Each signal domain specifies a particular destination in the cell for the precursor protein.

The term “region” refers to a physically contiguous portion of the primary structure of a biomolecule. In the case of proteins, a region is defined by a contiguous portion of the amino acid sequence of that protein. In some embodiments a “region” is associated with a function of the biomolecule.

The term “fragment” as used herein refers to a physically contiguous portion of the primary structure of a biomolecule. In the case of proteins, a portion is defined by a contiguous portion of the amino acid sequence of that protein and refers to at least 3-5 amino acids, at least 8-10 amino acids, at least 11-15 amino acids, at least 17-24 amino acids, at least 25-30 amino acids, and at least 30-45 amino acids. In the case of oligonucleotides, a portion is defined by a contiguous portion of the nucleic acid sequence of that oligonucleotide and refers to at least 9-15 nucleotides, at least 18-30 nucleotides, at least 33-45 nucleotides, at least 48-72 nucleotides, at least 75-90 nucleotides, and at least 90-130 nucleotides. In some embodiments, portions of biomolecules have a biological activity. In the context of the present invention, PIP polypeptide fragments do not comprise the entire PIP polypeptide sequence set forth in SEQ ID NO:1.

A “native sequence” polypeptide is one that has the same amino acid sequence as a polypeptide derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other species.

As used herein, the phrase “homologous nucleotide sequence,” or “homologous amino acid sequence,” or variations thereof, refers to sequences characterized by a homology, at the nucleotide level or amino acid level, of at least a specified percentage and is used interchangeably with “sequence identity”. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. Homologous amino acid sequences include those amino acid sequences which contain conservative amino acid substitutions and polypeptides that have the same or similar binding and/or activity. In some embodiments, a nucleotide or amino acid sequence is homologous if it has at least 60%, 70%, 80%, 85%, 90%, 95%, 96%, 97%, 98%, or 99% identity. In some embodiments, a nucleotide or amino acid sequence is homologous if it has 1-10, 10-20, 20-30, 30-40, 40-50, or 50-60 nucleotide/amino acid substitutions, additions, or deletions. In some embodiments, the homologous amino acid sequences have no more than 5 or no more than 3 conservative amino acid substitutes.

Percent homology or identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., (1981), 2:482). In some embodiments, homology between the probe and target is between about 75% to about 85%. In some embodiments, nucleic acids have nucleotides that are at least about 95%, about 97%, about 98%, about 99% and about 100% homologous to SEQ ID NO:2, or a portion thereof.

Homology may also be at the polypeptide level. In some embodiments, polypeptides are about 95%, about 97%, about 98%, about 99% and about 100% homologous to SEQ ID NO:1, or a portion thereof.

As used herein, the term “mixing” refers to the process of combining one or more compounds, cells, molecules, and the like together in the same area. This may be performed, for example, in a test tube, petri dish, or any container that allows the one or more compounds, cells, or molecules, to be intermingled.

As used herein, the term “substantially purified” refers to a compound (e.g., either a polynucleotide or a polypeptide or an antibody) that is removed from its natural environment and is at least 60% free, at least 75% free, and at least 90% free from other components with which it is naturally associated.

The term “pharmaceutically acceptable carrier” refers to a carrier for administration of a therapeutic agent, such as antibodies, polypeptides, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

The pharmaceutical compositions of the PIP proteins, PIP fusion proteins and PIP analogs of the methods of the present invention may be administered by any means that achieve the generally intended purpose: to treat T2DM, pancreatic beta cell impairment, glucose intolerance, hyperglycemia, insulin resistance, obesity, dyslipidemia, NASH, metabolic syndrome, and other metabolic syndrome or critically ill patients. The term “parenteral” as used herein refers to modes of administration that include intravenous, intramuscular, intraperitoneal, intrasternal, subcutaneous, and intraarticular injection and infusion. The dosage administered will be dependent upon the age, health, and weight of the recipient, kind of concurrent treatment, if any, frequency of treatment, and the nature of the effect desired. Compositions within the scope of the invention include all compositions wherein a PIP protein, PIP fusion protein or PIP analog is present in an amount that is effective to achieve the desired medical effect for treatment of the metabolic disorders listed herein, including but not limited to T2DM, pancreatic beta cell impairment, glucose intolerance, hyperglycemia, insulin resistance, obesity, dyslipidemia, NASH, metabolic syndrome, and other metabolic syndrome. While individual needs may vary from one patient to another, the determination of the optimal ranges of effective amounts of all of the components is within the ability of the clinician of ordinary skill.

The PIP proteins, PIP fusion proteins and PIP analogs of the methods of the present invention can be formulated according to known methods to prepare pharmaceutically useful compositions. A desired formulation would be one that is a stable lyophilized product that is reconstituted with an appropriate diluent or an aqueous solution of high purity with optional pharmaceutically acceptable carriers, preservatives, excipients or stabilizers (Remington's Pharmaceutical Sciences 16th edition 1980). The variants of the present invention may be combined with a pharmaceutically acceptable buffer, and the pH adjusted to provide acceptable stability, and a pH acceptable for administration.

For parenteral administration, in one embodiment, the PIP proteins and PIP fusion proteins are formulated generally by mixing one or more of them at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. Preferably, one or more pharmaceutically acceptable anti-microbial agents may be added. Phenol, m-cresol, and benzyl alcohol are preferred pharmaceutically acceptable anti-microbial agents.

Optionally, one or more pharmaceutically acceptable salts may be added to adjust the ionic strength or tonicity. One or more excipients may be added to further adjust the tonicity of the formulation. Glycerin, sodium chloride, and mannitol are examples of a tonicity-adjusting excipients.

The therapeutic compositions employed by the methods of the present invention, and/or comprising the PIP, PIP analog, or PIP fusion protein of the invention, may be used as a regularly administered (e.g., daily, more preferably weekly, biweekly, or monthly) injectable, either alone or in combination with oral anti-diabetic agents, which will improve the glycemic control.

Those skilled in the art can readily optimize pharmaceutically effective dosages and administration regimens for therapeutic compositions comprising a PIP, PIP analog, or PIP fusion protein, as determined by good medical practice and the clinical condition of the individual patient. A typical dose range for a PIP, PIP analog, or PIP fusion proteins of the methods of the present invention will range from about 0.01 mg per day to about 1000 mg per day (or about 0.05 mg per week to about 5000 mg per week administered once per week, bi-weekly, or monthly) for an adult. Preferably, the dosage ranges from about 0.1 mg per day to about 100 mg per day (or about 0.5 mg per week to about 500 mg per week administered once per week, bi-weekly, or monthly), more preferably from about 1.0 mg/day to about 10 mg/day (or about 5 mg per week to about 50 mg per week administered once per week, bi-weekly, or monthly).

Most preferably, the dosage is about 1-5 mg/day (or about 5 mg per week to about 25 mg per week administered once per week, bi-weekly, or monthly).

The appropriate dose of a PIP, PIP analog, or PIP fusion protein administered will result in improved beta cell function, improved insulin sensitivity and lower blood glucose levels, and thus is useful for treating the metabolic disorders listed herein, including but not limited to T2DM, pancreatic beta cell impairment, glucose intolerance, hyperglycemia, insulin resistance, obesity, dyslipidemia, NASH, metabolic syndrome, and other metabolic disorders.

Having now described the present invention in detail, the same will be more clearly understood by reference to the following examples, which are included herewith for purposes of illustration only and are not intended to be limiting of the invention.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); Handbook of Experimental Immunology, Vols. I-IV (D. M. Weir and C. C. Blackwell, eds., 1986, Blackwell Scientific Publications); and Sambrook et al., Molecular Cloning: A Laboratory Manual (2nd Edition, 1989).

### PIP Variants

Although the PIP peptides, polypeptides, proteins, analogs, variants, and related fusion proteins and pharmaceutical compositions embodied by the present methods and compositions of the invention are intended to maximize the compositions' physical and chemical stability under both physiological and preserved pharmaceutical formulation conditions, maintaining, or even enhancing, the biological potency of said compositions as compared to, e.g., wild-type PIP, is an important consideration as well. Therefore, the biological potency of the compositions of the present invention is defined by the ability of the proteins to improve insulin secretion, improve insulin sensitivity and/or improve glucose control, as shown herein in the examples.

The proteins, polypeptides, peptides, variants, analogs, fusions and pharmaceutical compositions of the invention administered according to this invention may be generated and/or isolated by any means known in the art. The most preferred method for producing the agent is through recombinant DNA methodologies and is well known to those skilled in the art. Such methods are described in Current Protocols in Molecular Biology (John Wiley & Sons, Inc., 2003), which is incorporated herein by reference.

The preferred embodiments include at minimum a biologically active protein, polypeptide, peptide, variant, analog, or fusion protein that retains PIP biological activity. Such an agent may contain at least one of the substitutions described and the composition of matter will possess biological activity. For instance, preferred truncations of the invention, and preferred substitution variants, possess PIP biological activity because they retain one or more biologically active peptides of the wild type PIP, or are similar enough (e.g., via conservative substitutions) to still possess PIP biological activity in spite of sequence difference(s).

In certain preferred embodiments, the compositions of the invention and the methods employing them possess similar or enhanced PIP biological activity (e.g., the ability to improve insulin secretion, and/or to improve insulin sensitivity, and/or to improve glucose control) relative to its wild-type PIP counterpart. Said compositions also may demonstrate other enhanced characteristics, such as increased half-life, as compared to the wild-type PIP.

Non-limiting examples of biologically active peptides of PIP, one or more of which are retained by the compositions of the present invention (either as exactly the same sequence, or as a variant thereof that possesses the same or improved biological activity), include peptides with improved properties of the molecule such as half-life extension.

The peptide may be produced by any and all means known to those skilled in the art, examples of which are included, but are not limited to enzymatic digestion, chemical synthesis or recombinant DNA methodologies.

The invention also encompasses polynucleotides encoding the above-described variants that may be in the form of RNA or in the form of DNA, which includes cDNA, genomic DNA, and synthetic DNA. The DNA may be double-stranded or single-stranded. The coding sequences that encode the proteins of the present invention may vary as a result of the redundancy or degeneracy of the genetic code.

The polynucleotides that encode for the proteins of the invention may include the following: only the coding sequence for the wild-type and its variant, the coding sequence for the variant and additional coding sequence such as a functional polypeptide, or a leader or signal sequence or a pro-protein sequence; the coding sequence for the variant and non-coding sequence, such as introns or non-coding sequence 5′ and/or 3′ of the coding sequence for the variant. Thus the term “polynucleotide encoding a variant” encompasses a polynucleotide that may include not only coding sequence for the variant but also a polynucleotide, which includes additional coding and/or non-coding sequence.

The invention further relates to variants of the described polynucleotides that encode for fragments, analogs and derivatives of the polypeptide that contain the indicated substitutions. The variant of the polynucleotide may be a naturally occurring allelic variant of the human PIP sequence, a non-naturally occurring variant, or a truncated variant as described above. Thus, the present invention also includes polynucleotides encoding the variants described above, as well as variants of such polynucleotides that encode for a fragment, derivative or analog of the disclosed variant. Such nucleotide variants include deletion variants, substitution variants, truncated variants, and addition or insertion variants as long as at least one of the indicated amino acid substitutions of the first or second embodiments is present.

The polynucleotides of the invention will be expressed in hosts after the sequences have been operably linked (i.e., positioned to ensure the functioning of) to an expression control sequence. These expression vectors are typically replicable in the host organisms either as episomes or as an integral part of the host chromosomal DNA. Commonly, expression vectors will contain selection markers, e.g., tetracycline, neomycin, or dihydrofolate reductase, to permit detection of those cells transformed with the desired DNA sequences. The PIP variant can be expressed in mammalian, insect, yeast, bacterial or other cells under the control of appropriate promoters. Cell free translation systems can also be employed to produce such proteins using RNAs derived from DNA constructs of the present invention.

Escherichia coli is a prokaryotic host useful particularly for cloning the polynucleotides of the present invention. Other microbial hosts suitable for use include Bacillus subtilus, Salmonella typhimurium, and various species of Serratia, Pseudomonas, Streptococcus, and Staphylococcus, although others may also be employed as a matter of choice. In these prokaryotic hosts, one can also make expression vectors, which will typically contain expression control sequences compatible with the host cell (e.g., an origin of replication). In addition, any of a number of well-known promoters may be present, such as the lactose promoter system, a tryptophan (Trp) promoter system, a beta-lactamase promoter system, or a promoter system from phages lambda or T7. The promoters will typically control expression, optionally with an operator sequence, and have binding site sequences and the like, for initiating and completing transcription and translation.

One skilled in the art of expression of proteins will recognize that methionine or methionine-arginine sequence can be introduced at the N-terminus of the mature sequence for expression in E. coli and are contemplated within the context of this invention. Thus, unless otherwise noted, proteins of the present invention expressed in E. coli have a methionine introduced at the N-terminus.

Other microbes, such as yeast or fungi, may also be used for expression. Pichia pastoris, Saccharomyces cerevisiae, Schizosaccharomyces pombe, and Pichia angusta are examples of preferred yeast hosts, with suitable vectors having expression control sequences, such as promoters (including that from 3-phosphoglycerate kinase or other glycolytic enzymes) an origin of replication, termination sequences and the like as desired. Aspergillus niger, Trichoderma reesei; and Schizophyllum commune, are examples of fungi hosts, although others may also be employed as a matter of choice.

Mammalian tissue cell culture may also be used to express and produce the polypeptides of the present invention. Eukaryotic cells are preferred, because a number of suitable host cell lines capable of secreting intact variants have been developed in the art, and include the CHO cell lines, various COS cell lines, NSO cells, Syrian Hamster Ovary cell lines, HeLa cells, or human embryonic kidney cell lines (i.e. HEK293, HEK293EBNA).

Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from SV40, adenovirus, bovine papilloma virus, cytomegalovirus, Rous sarcoma virus, and the like. Preferred polyadenylation sites include sequences derived from SV40 and bovine growth hormone.

The vectors containing the polynucleotide sequences of interest (e.g., that encode the proteins of the invention and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for the compositions of the present invention.

The proteins, polypeptides, and/or peptides of the invention should be formulated and dosed in a fashion consistent with good medical practice, taking into account the clinical condition of the patient, the site of delivery of the protein compositions, the method of administration, the scheduling of administration, and other factors known to practitioners. The “therapeutically effective amount” of the proteins of the invention for purposes herein is thus determined by such considerations.

The pharmaceutical compositions of the proteins of the present invention may be administered by any means that achieve the generally intended purpose: to treat T2DM, dyslipidemia, NASH, insulin resistance, hyperinsulinemia, glucose intolerance, hyperglycemia, obesity, metabolic syndrome, and other metabolic disorders. Non-limiting permissible means of administration include, for example, by inhalation, suppository, application to mucosal tissue (such as by lavage to vaginal, rectal, urethral, buccal or sublingual tissue), orally, nasally, topically, intranasally, intraperitoneally, parenterally, intravenously, intramuscularly, intrasternally, by intraarticular injection, intralymphatically, interstitially, intra-arterially, subcutaneously, intrasynovial, transepithelial, and transdermally. In some embodiments, the pharmaceutical compositions are administered by lavage, orally or inter-arterially. Other suitable methods of introduction can also include rechargeable or biodegradable devices and slow or sustained release polymeric devices. The pharmaceutical compositions of this invention can also be administered as part of a combinatorial therapy with other known therapeutic agents, especially those that improve metabolic disorders.

The dosage administered will be dependent upon the age, health, and weight of the recipient, kind of concurrent treatment, if any, frequency of treatment, and the nature of the effect desired. Compositions within the scope of the invention include all compositions wherein a PIP variant is present in an amount that is effective to achieve the desired medical effect for treatment of T2DM, glucose intolerance, pancreatic beta cell impairment, hyperglycemia, insulin resistance, obesity, dyslipidemia, NASH, metabolic syndrome, and other metabolic disorders. While individual needs may vary from one patient to another, the determination of the optimal ranges of the effective amount is within the ability of the clinician of ordinary skill.

The compositions of the present invention can be formulated according to known methods to prepare pharmaceutically useful compositions. A desired formulation would be one that is a stable lyophilized product that is reconstituted with an appropriate diluent or an aqueous solution of high purity with optional pharmaceutically acceptable carriers, preservatives, excipients or stabilizers [Remington's Pharmaceutical Sciences 16th edition (1980)]. The proteins of the present invention may be combined with a pharmaceutically acceptable buffer, and the pH adjusted to provide acceptable stability, and a pH acceptable for administration.

For parenteral administration, in one embodiment, the proteins of the invention are formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. Preferably, one or more pharmaceutically acceptable anti-microbial agents may be added. Phenol, m-cresol, and benzyl alcohol are preferred pharmaceutically acceptable anti-microbial agents.

Optionally, one or more pharmaceutically acceptable salts may be added to adjust the ionic strength or tonicity. One or more excipients may be added to further adjust the tonicity of the formulation. Glycerin, sodium chloride, and mannitol are examples of an tonicity-adjusting excipient.

Those skilled in the art can readily optimize pharmaceutically effective dosages and administration regimens for therapeutic compositions comprising compositions of the present invention, as determined by good medical practice and the clinical condition of the individual patient. A typical dose range for the compositions of the present invention will range from about 0.01 mg per day to about 1000 mg per day (or about 0.05 mg per week to about 5000 mg per week administered once per week, bi-weekly, or monthly) for an adult. Preferably, the dosage ranges from about 0.1 mg per day to about 100 mg per day (or about 0.5 mg per week to about 500 mg per week administered once per week, bi-weekly, or monthly), more preferably from about 1.0 mg/day to about 10 mg/day (or about 5 mg per week to about 50 mg per week administered once per week, bi-weekly, or monthly). Most preferably, the dosage is about 1-5 mg/day (or about 5 mg per week to about 25 mg per week administered once per week, bi-weekly, or monthly). The appropriate dose of PIP, a PIP analog, or PIP fusion protein administered will result in lowering blood glucose levels, and thus is useful for treating the metabolic disorders listed herein, including but not limited to T2DM, obesity and metabolic disorders.

In addition, because hyperglycemia and insulin resistance are common in critically ill patients given nutritional support, some intensive care units (ICUs) administer insulin to treat hyperglycemia in fed critically ill patients. In fact, recent studies document the use of exogenous insulin to maintain blood glucose at a level no higher than 110 mg per deciliter reduced morbidity and mortality among critically ill patients in the surgical ICU, regardless of whether they had a history of diabetes (Van den Berghe, et al. (2001) N Engl J Med., 345:1359). Thus, proteins of the present invention are uniquely suited to help restore metabolic stability in metabolically unstable critically ill patients. Proteins of the invention such as those containing variants of PIP are unique in that they improve glucose tolerance and enhances insulin secretion, improves insulin sensitivity but do not induce hypoglycemia.

In another aspect of the present invention, proteins of the invention for use as a medicament for the treatment of T2DM, glucose intolerance, pancreatic beta cell impairment, hyperglycemia, insulin resistance, obesity, dyslipidemia, NASH, metabolic syndrome, and other metabolic disorders is contemplated.

### Site-Specific PIP Mutants

In some embodiments, the fusion proteins of the present invention include mutants of GLP-1, FGF21, or Exendin-4 (mutations convey DPP-4 resistance, are for pegylation, or for other purposes).

In some embodiments, the proteins of the invention comprise proteins that have PIP activity, such as: (i) wild type PIP with additional disulfides, unnatural amino acids, or modifications to promote dimerization such as formation of a disulfide bond or introduction of a cysteine at another site, or dimerization through a fused Fc domain, or dimer formation through a cross-linker such as a bifunctional PEG;

(ii) fragments of PIP;

(iii) proteins selected to have PIP activity; or

(iv) a PIP mimetic antibody (of various formats such as Fab, unibody, svFc etc.).

In some embodiments, the fusion proteins of the invention comprise one or more of the following linkers: a simple amide bond, short peptides (particularly Ser/Gly repeats), additional residues from the PIP translated sequence, or a larger linker up to an entire protein (such as an Fc domain, an HSA-binding helix bundle, HSA, etc.). The two moieties can also be linked by other chemical means, such as through unnatural amino acids or standard chemical linkers (maleimide-Cys, NHS-Lys, “click chemistry”, etc.).

In some embodiments, the fusion protein of the invention comprises PEGylation occurring at one, two, or more specific sites. In preferred embodiments, the PEGylation occurs within the PIP molecule or the linker. In some embodiments, the PEGylation is not within ˜10 amino acids of the N-term of PIP. PEGylation attachment chemistries can include NHS-Lys, maleimide-Cys, unnatural amino acids (aldehyde, “click chemistry”, Pcl, etc.) and can be combined with suitable protein variants to control the stoichiometry of the reaction.

The PEG group of the fusion proteins of the invention can be of any size (e.g., 1, 2, 3, 4, 5, 10, 20, 24, 29, 30, 40 kDa), and can be linear, branched, or comb structured, with a preference for a total PEGylation of greater than or equal to 40 kDa. Optimal PEGylation achieves half-life extension sufficient for once weekly dosing. PEGylation of protein dimers, trimers, tetramers etc. may result in adequate serum half-life extension using shorter PEG polymers.

Preferred half-life extension methodologies for the fusion proteins of the invention include integrating an IgG Fc domain or HSA into the linker and may not require PEGylation. Additionally, using Fc domain fusions will result in dimerization and may result in enhanced potency in addition to half-life extension.

In certain embodiments of the invention, other attachments may be made to proteins, polypeptides, and/or peptides of the invention, to achieve half-life extension and other improved biological properties. They can include attaching PEG-cholesterol conjugates (including micelles and liposomes) to the proteins, polypeptides, and/or peptides of the invention, and/or attaching sugars (glycosylate) to the proteins, polypeptides, and/or peptides of the invention. In still other embodiments, similar techniques are employed to add such conjugates as polysialic acid (PSA), hydroxyethyl starch (HES), albumin-binding ligands, or carbohydrate shields to proteins, polypeptides, and/or peptides.

The HESylation technique, for example, couples branched HES chains (60 kDa or 100 kDa, highly branched amylopectin fragments from corn starch) to proteins, polypeptides, and/or peptides via reductive alkylation. Polysialation conjugates proteins, polypeptides, and/or peptides of interest with PSA polymers in a manner similar to PEGylation. PSA polymers are negatively charged, non-immunogenic polymers that occur naturally in the body and are available in molecular weights of 10-50 kDa.

### Chemically-Modified Dual Function Protein Mutants

Chemically modified forms of the fusion proteins described herein, including, e.g., truncated and variant forms of the PIP fusions described herein, can be prepared by one skilled in the art, given the disclosures described herein. Such chemically modified dual function proteins are altered such that the chemically modified mutant is different from the unmodified mutant, either in the type or location of the molecules naturally attached to the mutant. Chemically modified mutants can include molecules formed by the deletion of one or more naturally-attached chemical groups.

In one embodiment, proteins of the present invention can be modified by the covalent attachment of one or more polymers. For example, the polymer selected is typically water-soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable. Non-water soluble polymers conjugated to proteins of the present invention also form an aspect of the invention.

Exemplary polymers each can be of any molecular weight and can be branched or unbranched. The polymers each typically have an average molecular weight of between about 2 kDa to about 100 kDa (the term “about” indicating that in preparations of a water-soluble polymer, some molecules will weigh more and some less than the stated molecular weight). The average molecular weight of each polymer is preferably between about 5 kDa and about 50 kDa, more preferably between about 12 kDa and about 40 kDa, and most preferably between about 20 kDa and about 35 kDa.

Suitable water-soluble polymers or mixtures thereof include, but are not limited to, N-linked or O-linked carbohydrates, sugars, phosphates, PEG (including the forms of PEG that have been used to derivatize proteins, including mono-(C1-C10), alkoxy-, or aryloxy-PEG), monomethoxy-PEG, dextran (such as low molecular weight dextran of, for example, about 6 kD), cellulose, or other carbohydrate based polymers, poly-(N-vinyl pyrrolidone) PEG, propylene glycol homopolymers, polypropylene oxide/ethylene oxide co-polymers, polyoxyethylated polyols (e.g., glycerol), and polyvinyl alcohol. Also encompassed by the present invention are bifunctional crosslinking molecules that can be used to prepare covalently attached PIP variant multimers. Also encompassed by the present invention are PIP mutants covalently attached to polysialic acid.

In some embodiments of the present invention, a PIP mutant is covalently, or chemically, modified to include one or more water-soluble polymers, including, but not limited to, PEG, polyoxyethylene glycol, or polypropylene glycol. See, e.g., U.S. Pat. Nos. 4,640,835; 4,496,689; 4,301,144; 4,670,417; 4,791,192; and 4,179,337. In some embodiments of the present invention, a PIP mutant comprises one or more polymers, including, but not limited to, monomethoxy-PEG, dextran, cellulose, another carbohydrate-based polymer, poly-(N-vinyl pyrrolidone)-PEG, propylene glycol homopolymers, a polypropylene oxide/ethylene oxide co-polymer, polyoxyethylated polyols (e.g., glycerol), polyvinyl alcohol, or mixtures of such polymers.

In some embodiments of the present invention, a PIP mutant is covalently-modified with PEG subunits. In some embodiments, one or more water-soluble polymers are bonded at one or more specific positions (for example, at the N-terminus) of the PIP mutant. In some embodiments, one or more water-soluble polymers are randomly attached to one or more side chains of a PIP mutant. In some embodiments, PEG is used to improve the therapeutic capacity of a PIP mutant. Certain such methods are discussed, for example, in U.S. Pat. No. 6,133,426, which is hereby incorporated by reference for any purpose.

In embodiments of the present invention wherein the polymer is PEG, the PEG group can be of any convenient molecular weight, and can be linear or branched. The average molecular weight of the PEG group will preferably range from about 2 kDa to about 100 kDa, and more preferably from about 5 kDa to about 50 kDa, e.g., 10, 20, 30, 40, or 50 kDa. The PEG groups will generally be attached to the PIP mutant via acylation or reductive alkylation through a reactive group on the PEG moiety (e.g., an aldehyde, amino, thiol, or ester group) to a reactive group on the PIP mutant (e.g., an aldehyde, amino, or ester group).

Branched PEG derivatives, also known as “Y-shaped” PEG derivatives, contain two linear methoxy PEG chain attached to a central core. The sterically bulky structure of these “Y-shaped” PEG derivatives will facilitate the single point attachment of the modified molecules. By way of example, three kinds of “Y-shaped” PEG derivatives are Y-NHS-40K (useful for amine PEGylation); Y-MAL-40K (useful for thiol PEGylation); and Y-ALD-40K (e.g., Y-AALD-40K and Y-PALD-40K; useful for N-terminal PEGylation). For amine PEGylation, the “Y-shape” NHS ester will react with the amino group of lysine(s) or the N-terminal amine in biological active molecules to produce a stable amide linkage(s). This NHS ester will couple with the targeted molecules at pH 7-8.5. For thiol PEGylation, the “Y-shape” maleimide will react with the thiol groups in biological active molecules to generate a stable 3-thiosuccinimidyl ether linkage. This maleimide will couple with the targeted molecules at an approximate pH of 7.4 in the presence of other functional groups. For N-terminal PEGylation, the “Y-shape” aldehyde will preferably react with the N-terminal amine in biologically active molecules to produce a stable amine linkage in the presence of a reducing reagent such as sodium cyanoborohydride. This aldehyde will couple with the N-terminal amine of the targeted molecules at pH 5-8. Reagents for performing branched PEGylation are available through, e.g., JenKem Technology.

The PEGylation of a polypeptide, including the proteins of the invention, can be specifically carried out using any of the PEGylation reactions known in the art. Such reactions are described, for example, in the following references: Francis et al., (1992), Focus on Growth Factors 3: 4-10; European Patent Nos. 0 154 316 and 0 401 384; and U.S. Pat. No. 4,179,337. For example, PEGylation can be carried out via an acylation reaction or an alkylation reaction with a reactive PEG molecule (or an analogous reactive water-soluble polymer) as described herein. For the acylation reactions, a selected polymer should have a single reactive ester group. For reductive alkylation, a selected polymer should have a single reactive aldehyde group. A reactive aldehyde is, for example, PEG propionaldehyde, which is water stable, or mono C1-C10 alkoxy or aryloxy derivatives thereof (see, e.g., U.S. Pat. No. 5,252,714).

In some embodiments of the present invention, a useful strategy for the attachment of the PEG group to a polypeptide involves combining, through the formation of a conjugate linkage in solution, a peptide and a PEG moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water-soluble polymer that can be used for protein modification. Therefore, the proteins of the invention fused to a polysaccharide polymer form embodiments of the present invention. Dextrans are polysaccharide polymers comprised of individual subunits of glucose predominantly linked by alpha 1-6 linkages. The dextran itself is available in many molecular weight ranges, and is readily available in molecular weights from about 1 kDa to about 70 kDa. Dextran is a suitable water-soluble polymer for use as a vehicle by itself or in combination with another vehicle (e.g., Fc). See, e.g., International Publication No. WO 96/11953. The use of dextran conjugated to therapeutic or diagnostic immunoglobulins has been reported. See, e.g., European Patent Publication No. 0 315 456, which is hereby incorporated by reference. The present invention also encompasses the use of dextran of about 1 kDa to about 20 kDa.

In general, chemical modification can be performed under any suitable condition used to react a protein with an activated polymer molecule. Methods for preparing chemically modified polypeptides will generally comprise the steps of: (a) reacting the polypeptide with the activated polymer molecule (such as a reactive ester or aldehyde derivative of the polymer molecule) under conditions whereby a PIP variant becomes attached to one or more polymer molecules, and (b) obtaining the reaction products. The optimal reaction conditions will be determined based on known parameters and the desired result. For example, the larger the ratio of polymer molecules to protein, the greater the percentage of attached polymer molecule. In one embodiment of the present invention, chemically modified PIP mutants can have a single polymer molecule moiety at the amino-terminus (see, e.g., U.S. Pat. No. 5,234,784)

Generally, conditions that can be alleviated or modulated by the administration of the present chemically modified PIP mutants include those described herein for proteins of the invention. However, the chemically modified PIP mutants disclosed herein can have additional activities, enhanced or reduced biological activity, or other characteristics, such as increased or decreased half-life, as compared to unmodified PIP mutants.

### PIP Fusion Proteins

As used herein, the term “PIP fusion polypeptide” or “PIP fusion protein” refers to a fusion of one or more amino acid residues (such as a heterologous protein or peptide) at the N-terminus or C-terminus of any PIP, PIP analog or fusion protein described herein.

Heterologous peptides and polypeptides include, but are not limited to, an epitope to allow for the detection and/or isolation of a PIP, PIP analog or fusion protein; a transmembrane receptor protein or a portion thereof, such as an extracellular domain or a transmembrane and intracellular domain; a ligand or a portion thereof which binds to a transmembrane receptor protein; an enzyme or portion thereof that is catalytically active; a polypeptide or peptide which promotes oligomerization, such as a leucine zipper domain; a polypeptide or peptide that increases stability, such as an immunoglobulin constant region; a functional or non-functional antibody, or a heavy or light chain thereof; and a polypeptide that has an activity, such as a therapeutic activity, different from the PIP or PIP analog of the methods of the present invention. Also encompassed by the present invention are PIP or PIP analogs fused to HSA.

PIP fusion proteins can be made by fusing heterologous sequences at either the N-terminus or at the C-terminus of a PIP or PIP analog. As described herein, a heterologous sequence can be an amino acid sequence or a non-amino acid-containing polymer. Heterologous sequences can be fused either directly to the PIP or PIP analog or via a linker or adapter molecule. A linker or adapter molecule can be one or more amino acid residues, e.g., 1, 2, 3, 4, 5, 6, 7, 8, or 9 residues, preferably from 10 to 50 amino acid residues, e.g., 10, 11, 12, 13, 14, 15, 16, 17, 18, 19, 20, 25, 30, 35, 40, 45, or 50 residues, and more preferably from 15 to 35 amino acid residues. A linker or adapter molecule can also be designed with a cleavage site for a DNA restriction endonuclease or for a protease to allow for the separation of the fused moieties.

a. Human Serum Albumin (HSA) and Fc Fusions

In one embodiment of the present invention, PIP or a PIP analog is fused to one or more domains of HSA or the Fc region of human IgG. Antibodies comprise two functionally independent parts, a variable domain known as “Fab,” that binds an antigen, and a constant domain known as “Fc,” or “HSA” that is involved in effector functions such as complement activation and attack by phagocytic cells. An Fc or HSA has a long serum half-life, whereas a Fab is short-lived (Capon et al., 1989, Nature 337: 525-31). When joined together with a therapeutic protein, an Fc or HSA domain can provide longer half-life or incorporate such functions as Fc or HSA receptor binding, protein A binding, complement fixation, and perhaps even placental transfer (Capon et al., (1989), Nature 337:525).

The resulting PIP or PIP analog fusion protein can be purified, for example, by the use of a Protein A affinity column. Peptides and proteins fused to an Fc or HSA region have been found to exhibit a substantially greater half-life in vivo than the unfused counterpart. Also, a fusion to an Fc region allows for dimerization/multimerization of the fusion polypeptide. The Fc or HSA region can be a naturally occurring Fc or HSA region, or can be altered to improve certain qualities, such as therapeutic qualities, circulation time, or reduced aggregation.

Useful modifications of protein therapeutic agents by fusion with the “Fc” domain of an antibody are discussed in detail in International Publication No. WO 00/024782, which is hereby incorporated by reference in its entirety. This document discusses linkage to a “vehicle” such as PEG, dextran, or an Fc region.

b. Fusion Protein Linkers

When forming the fusion proteins of the present invention, a linker can, but need not, be employed. When present, the linker's chemical structure may not critical, since it serves primarily as a spacer. The linker can be made up of amino acids linked together by peptide bonds. In some embodiments of the present invention, the linker is made up of from 1 to 20 amino acids linked by peptide bonds, wherein the amino acids are selected from the 20 naturally occurring amino acids. In various embodiments, the 1 to 20 amino acids are selected from the amino acids glycine, serine, alanine, proline, asparagine, glutamine, and lysine. In some embodiments, a linker is made up of a majority of amino acids that are sterically unhindered, such as glycine and alanine. In some embodiments, linkers are polyglycines, polyalanines, combinations of glycine and alanine (such as poly(Gly-Ala)), or combinations of glycine and serine (such as poly(Gly-Ser)).

The linkers described herein are exemplary, and linkers that are much longer and which include other residues are contemplated by the present invention. Non-peptide linkers are also contemplated by the present invention. For example, alkyl linkers can be used. These alkyl linkers can further be substituted by any sterically non-hindering group, including, but not limited to, a lower alkyl (e.g., C1-C6), lower acyl, halogen (e.g., Cl, Br), CN, NH2, or phenyl. An exemplary non-peptide linker is a PEG linker, wherein the linker has a molecular weight of 100 to 5000 kDa, for example, 100 to 500 kDa.

c. Fusion Protein Partners

Other peptides or proteins that have glucose lowering effects, including but not limited to GLP-1, FGF21 and Exendin-4, can be fused to the PIP peptides, polypeptides, proteins, variants, and analogs comprising the methods and compositions of the present invention, using conventional techniques known in the art and described herein. Said GLP-1, FGF21, and Exendin-4 may be fused in their wild type form, or as biologically active truncations, variants, or analogs thereof.

GLP-1 is a potent metabolic regulator already represented in the clinic (Knudsen et al. (2004) Journal of Medicinal Chemistry 47, 4128). GLP-1 is a 36 amino acid incretin secreted by L-cells of the mammalian gut, acting on both alpha and beta cells to stimulate insulin secretion and inhibit glucagon release in a glucose-dependent manner (Hare et al. (2010) Diabetes 59, 1765; Meier et al. (2005) Diabetes-Metabolism Research and Reviews 21, 91). GLP-1 binds to and activates the GLP-1 receptor (GLP-1R), a seven-transmembrane helix protein of the class II family of G-protein coupled receptors (GPCRs) (Mayo et al. (2003) Pharmacological Reviews 55:167). As a GLP-1 receptor agonist, GLP-1 has an important role in decreasing post-prandial blood glucose levels by stimulating insulin secretion from the pancreas in order to increase glucose absorption in the peripheral tissues and inhibiting glucagon secretion, resulting in reduced hepatic glucose release.

Exendin-4 is a clinically important GLP-1 receptor agonist. Exendin-4 is a 39 residue polypeptide produced in the salivary glands of the Gila Monster lizard (Goke et al. (1993) Diabetes 46:433; Fehmann et al. (1995) Endocrine Rev. 16:390). Although it is the product of a non-mammalian gene and appears to be expressed only in the salivary gland, Exendin-4 shares a 52% amino acid sequence homology with GLP-1, and in mammals interacts with the GLP-1 receptor (Goke, et al.; Thorens et al. (1993) Diabetes 42:1678). In vitro, Exendin-4 has been shown to promote insulin secretion by insulin producing cells and, given in equimolar quantities, is more potent than GLP-1 at causing insulin release from insulin producing cells. Furthermore, Exendin-4 potently stimulates insulin release to reduce plasma glucose levels in both rodents and humans and is longer acting than GLP-1; however, because it does not occur naturally in mammals, Exendin-4 has certain potential antigenic properties in mammals that GLP-1 lacks.

The GLP-1 and Exendin-4 sequences with NCBI reference numbers NP_002045 and AAB22006.1, respectively, can be found in such patent publications as, e.g., WO98/19698 and WO87/06941A, assigned to Eli Lilly and Co. and the General Hospital Corp., respectively (GLP-1) and U.S. Pat. No. 5,424,286, assigned to Amylin (Exendin-4).

FGF21 has been shown to induce insulin-independent glucose uptake. FGF-21 has also been shown to ameliorate hyperglycemia in a range of diabetic rodent models. In addition, transgenic mice over-expressing FGF-21 were found to be resistant to diet-induced metabolic abnormalities, and demonstrated decreased body weight and fat mass, and enhancements in insulin sensitivity (Badman, M. K. et al. (2007) Cell Metab 5, 426).

Administration of FGF-21 to diabetic non-human primates caused a decline in fasting plasma glucose, triglycerides, insulin and glucagon levels, and led to significant improvements in lipoprotein profiles, including a nearly 80% increase in HDL cholesterol (Kharitonenkov, A. et al. (2007) Endocrinology 148: 774). Recent studies investigating the molecular mechanisms of FGF21 action have identified FGF21 as an important endocrine hormone that helps to control adaptation to the fasting state (Badman et al. (2009) Endocrinology 150: 4931; Inagaki et al. (2007) Cell Metabolism 5: 415). This provides a previously missing link downstream of PPARα, by which the liver communicates with the rest of the body in regulating the biology of energy homeostasis (Galman et al. (2008) Cell Metabolism 8: 169; Lundasen et al. (2007) Biochemical and Biophysical Research Communications 360: 437).

Non-limiting examples of FGF21 variants which comprise PIP fusions of the present invention, i.e., which comprise the compositions of matter and methods of the present invention, include those described in PCT publications WO2008/011633, WO12/066075, WO13/057371, and WO13/049247; U.S. Pat. No. 7,491,697, U.S. Pat. No. 7,576,190, U.S. Pat. No. 8,034,770, U.S. Pat. No. 8,188,040, U.S. Pat. No. 8,361,963, and U.S. Pat. No. 8,410,051; and US patent publications US2010/216715 and US2008/0255045, as well as any related patent family members of the above.

The PIP fusion proteins as described above, and as comprising the compositions of matter and methods of the present invention, e.g., which as comprise PIP fused to one or more of FGF21, GLP-1, and Exendin-4, which have improved pharmaceutical properties over the constituent agents under pharmaceutical formulation conditions, e.g., are more stable, possess the ability to improve metabolic parameters for subjects to whom they are administered, are less susceptible to proteolysis and enzymatic degradation, are less likely to aggregate and form complexes and are less likely to be immunogenic.

Therapeutic Compositions of PIP Proteins and PIP Fusion Proteins and Administration Thereof

Methods of treatment using therapeutic compositions comprising PIP proteins and PIP fusion proteins are within the scope of the present invention. Such pharmaceutical compositions can comprise a therapeutically effective amount of a PIP or fusion protein in admixture with a pharmaceutically or physiologically acceptable formulation agent selected for suitability with the mode of administration.

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or PEG), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides; preferably sodium or potassium chloride; or mannitol sorbitol), delivery vehicles, diluents, excipients and/or pharmaceutical adjuvants (see, e.g., Remington's Pharmaceutical Sciences (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage (see, e.g., Remington's Pharmaceutical Sciences). Such compositions can influence the physical state, stability, rate of in vivo release, and rate of in vivo clearance of the PIP or fusion protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the present invention, PIP or fusion protein compositions can be prepared for storage by mixing the selected composition having the desired degree of purity with optional formulation agents (Remington's Pharmaceutical Sciences, supra) in the form of a lyophilized cake or an aqueous solution. Further, the PIP or fusion protein product can be formulated as a lyophilizate using appropriate excipients such as sucrose.

The PIP or fusion protein pharmaceutical compositions can be selected for parenteral delivery. Alternatively, the compositions can be selected for inhalation, intranasal delivery, or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention can be in the form of a pyrogen-free, parenterally acceptable, aqueous solution comprising the desired PIP or fusion protein in a pharmaceutically acceptable vehicle. A particularly suitable vehicle for parenteral injection is sterile distilled water in which a PIP or fusion protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a PIP or fusion protein can be formulated as a dry powder for inhalation. PIP or fusion protein inhalation solutions can also be formulated with a propellant for aerosol delivery. In yet another embodiment, solutions can be nebulized. Pulmonary administration is further described in International Publication No. WO 94/20069, which describes the pulmonary delivery of chemically modified proteins.

It is also contemplated that certain formulations can be administered orally. In one embodiment of the present invention, PIP or fusion proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the PIP or fusion protein. Diluents, flavorings, low melting point waxes, vegetable oils, lubricants, suspending agents, tablet disintegrating agents, and binders can also be employed.

Another pharmaceutical composition can involve an effective quantity of PIP or fusion proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional PIP or fusion protein pharmaceutical compositions will be evident to those skilled in the art, including formulations involving PIP or fusion proteins in sustained- or controlled-delivery formulations. Techniques for formulating a variety of other sustained- or controlled-delivery means, such as liposome carriers, bio-erodible microparticles or porous beads and depot injections, are also known to those skilled in the art (see, e.g., International Publication No. WO 93/15722, which describes the controlled release of porous polymeric microparticles for the delivery of pharmaceutical compositions, and Wischke & Schwendeman, (2008), Int. J Pharm. 364: 298; Freiberg & Zhu, (2004), Int. J Pharm. 282:1), which discuss microsphere/microparticle preparation and use).

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et al., (1983), Biopolymers 22: 547), poly(2-hydroxyethyl-methacrylate) (Langer et al., (1981), J. Biomed. Mater. Res. 15: 167 and Langer, (1982), Chem. Tech. 12: 98), ethylene vinyl acetate or poly-D-3-hydroxybutyric acid (European Patent No. 0 133 988). Sustained-release compositions can also include liposomes, which can be prepared by any of several methods known in the art. See, e.g., Epstein et al., (1985), Proc. Natl. Acad. Sci. U.S.A. 82: 3688-92; and European Patent Nos. 0 036 676, 0 088 046, and 0 143 949.

The PIP or fusion protein pharmaceutical composition to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the present invention is directed to kits for producing a single-dose administration unit. The kits can each contain both a first container having a dried protein and a second container having an aqueous formulation. Also included within the scope of this invention are kits containing single and multi-chambered pre-filled syringes (e.g., liquid syringes and lyosyringes).

The effective amount of a PIP or fusion protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the PIP or fusion protein is being used, the route of administration, and the size (body weight, body surface, or organ size) and condition (the age and general health) of the patient. Accordingly, the clinician can titer the dosage and modify the route of administration to obtain the optimal therapeutic effect. A typical dosage can range from about 0.1 μg/kg to up to about 100 mg/kg or more, depending on the factors mentioned above. In other embodiments, the dosage can range from 0.1 μg/kg up to about 100 mg/kg; or 1 μg/kg up to about 100 mg/kg; or 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, up to about 100 mg/kg. In yet other embodiments, the dosage can be 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1000 μg/kg, 2000 μg/kg, 3000 μg/kg, 4000 μg/kg, 5000 μg/kg, 6000 μg/kg, 7000 μg/kg, 8000 μg/kg, 9000 μg/kg or 10 mg/kg.

The frequency of dosing will depend upon the pharmacokinetic parameters of the PIP or fusion protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems (which may also be injected); or by implantation devices. Where desired, the compositions can be administered by bolus injection or continuously by infusion, or by implantation device.

Alternatively or additionally, the composition can be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

### Therapeutic Uses of PIP Proteins, PIP Variants, and PIP Fusion Proteins

PIP protein, variants, or fusion proteins can be used to treat, diagnose, ameliorate, or prevent a number of diseases, disorders, or conditions, including, but not limited to metabolic disorders. In one embodiment, the metabolic disorder to be treated is diabetes, e.g., T2DM. In another embodiment, the metabolic disorder is obesity. Other embodiments include metabolic conditions or disorders such as T2DM, glucose intolerance, hyperglycemia, dyslipidemia, obesity, NASH, insulin resistance, metabolic syndrome, hypertension, cardiovascular disease, atherosclerosis, peripheral arterial disease, stroke, heart failure, coronary heart disease, kidney disease, diabetic complications, neuropathy, gastroparesis and other metabolic disorders.

A disorder or condition such as T2DM or obesity can be treated by administering a PIP protein, variant, or fusion protein as described herein to a patient in need thereof in the amount of a therapeutically effective dose. The administration can be performed as described herein, such as by IV injection, intraperitoneal injection, intramuscular injection, or subcutaneous injection. In most situations, a desired dosage can be determined by a clinician, as described herein, and can represent a therapeutically effective dose of the PIP protein, variant, or fusion protein. It will be apparent to those of skill in the art that a therapeutically effective dose of PIP protein, variant, or fusion protein will depend, inter alia, upon the administration schedule, the unit dose of antigen administered, whether the nucleic acid molecule or polypeptide is administered in combination with other therapeutic agents, the immune status and the health of the recipient. The term “therapeutically effective dose,” as used herein, means that amount of PIP protein, variant, or fusion protein that elicits the biological or medicinal response in a tissue system, animal, or human being sought by a researcher, medical doctor, or other clinician, which includes alleviation of the symptoms of the disease or disorder being treated.

### Pharmaceutical Compositions

The present invention also provides pharmaceutical compositions comprising one or more of the PIP or PIP fusion proteins or mutants described herein and a pharmaceutically acceptable carrier. In some embodiments the pharmaceutical compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

### Fusion Proteins and PIP-Derived Peptidic Compounds

In another embodiment, the PIP, PIP variant, or PIP compositions of the present invention can be made into a fusion protein or peptidic compound derived from the PIP, PIP variant, or PIP fusion protein amino acid sequences. Such fusion proteins and peptidic compounds can be made using standard techniques known in the art. For example, peptidic compounds can be made by chemical synthesis using standard peptide synthesis techniques and then introduced into cells by a variety of means known in the art for introducing peptides into cells (e.g., liposome and the like).

The in vivo half-life of the fusion protein or peptidic compounds of the invention can be improved by making peptide modifications, such as the addition of N-linked glycosylation sites into PIP, PIP variant, or PIP fusion proteins, or conjugating PIP, PIP variant, or PIP fusion proteins to PEG (pegylation), e.g., via lysine-monopegylation or cysteine-monopegylation. Such techniques have proven to be beneficial in prolonging the half-life of therapeutic protein drugs. It is expected that pegylation of the PIP, PIP variant, or PIP compositions of the invention may result in similar pharmaceutical advantages.

In addition, pegylation can be achieved in any part of a polypeptide of the invention by the introduction of a nonnatural amino acid. Certain nonnatural amino acids can be introduced by the technology described in Deiters et al., (2003) J Am Chem Soc 125:11782; Wang and Schultz, (2003) Science 301:964; Wang et al., (2001) Science 292:498; Zhang et al., Science (2004) 303:371 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The PIP or PIP fusion proteins containing these novel amino acids can then be pegylated at these chosen sites in the protein.

### EXAMPLES

### Example 1

### Effect of Roux-En-Y Gastric Bypass (RYGB) Surgery on Pancreatic Beta Cells in Zucker Diabetic Fatty (ZDF) and Goto-Kakizaki (GK) Rats

The aim of this study was to investigate the effects of RYGB surgery on pancreatic beta cell response in two rodent models of beta cell impairment, Zucker Diabetic fatty (ZDF) and Goto Kakizaki (GK) rats.

Bariatric surgeries such as RYGB have been shown to be associated with rapid body weight loss and near-complete resolution of T2DM (Ahn S M et al (2010), Ann NY Acad. Sci. 1212: E37). This resolution of diabetes is believed to be due a combination of improved peripheral insulin sensitivity and improved pancreatic beta cell function. Whereas most RYGB studies show normalization of blood glucose with improved insulin sensitivity and body weight loss (Chambers A P et al (2011), Gastroenterology, 141:950; Campos G M et al (2010), J gastrointest Surg 14: 15), the exact nature of the regulation of pancreatic beta cell function is comparatively less well studied. To examine the underlying mechanisms behind the beta cell function and glycemia following bariatric surgery, we performed a series of studies in rodent (obese and non-obese) models that underwent RYGB surgery. The figures show data from a study in ZDF rat, an obese insulin resistant diabetic beta cell-impaired model (Shimabukuro M et al (1998), Proc Natl Acad Sci USA 95: 2498), and in GK rat, a non-obese pre-diabetic polygenic beta cell-dysfunctional model (Portha B (2005), Diabetes Metab Res Rev 21: 495) to evaluate the impact of RYGB surgery on beta cell health and function. In addition, the study was performed such that collection of key tissues, such and pancreas and gastro-intestinal (GI) tract, could be carried out at different time points following RYGB surgery. The goal of the tissue collection was to conduct transcriptional profiling to discover novel factors and pathways that might mediate the changes in beta cell regulation at various time points following RYGB surgery.

Research Design and Methods

Rats (ZDF or GK, 10-12 weeks old at the start of the study; n=8-10/group/time point) were distributed by their body weight into 3 groups before RYGB surgery and were assigned as RYGB surgery rats, Sham-operated (laporotomy without surgery) Ad-libitum (Sham AL) that had free access to food and water, and Sham-operated pair-fed rats (Sham PF) that were matched with average food intake that was consumed by the RYGB-operated rats. The entire study was performed as a time-course with functional tests and tissues and plasma collection done at 3 time points after surgery. In vivo functional tests were conducted, and plasma, pancreas and different segments of GI tract were collected at 3, 6 and 12 or 15 weeks post-surgery. Beta cell function was measured by changes in plasma insulin levels following either an oral glucose challenge or an intraperitoneally (IP) administered glucose with arginine challenge Plasma gut hormones and body weights were measured to monitor the changes in the level of common biomarkers such as incretins that are shown to be associated with the surgery. Pancreatic beta cell regulation was evaluated by immunohistochemistry and measuring changes in insulin-positive vs. non-insulin positive staining of the pancreas excised from rats.

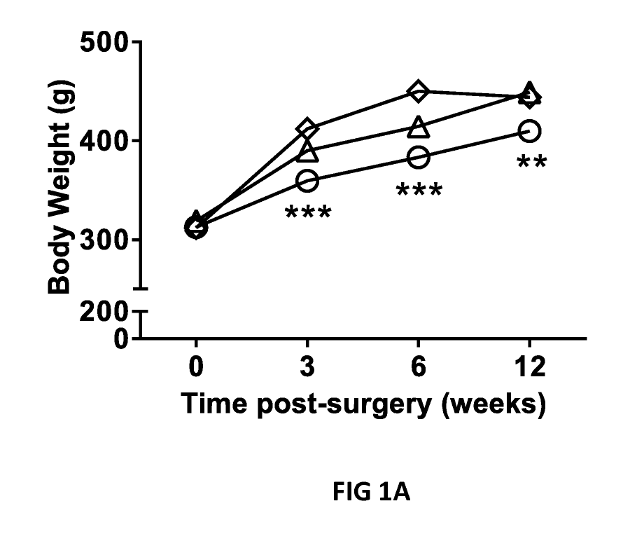

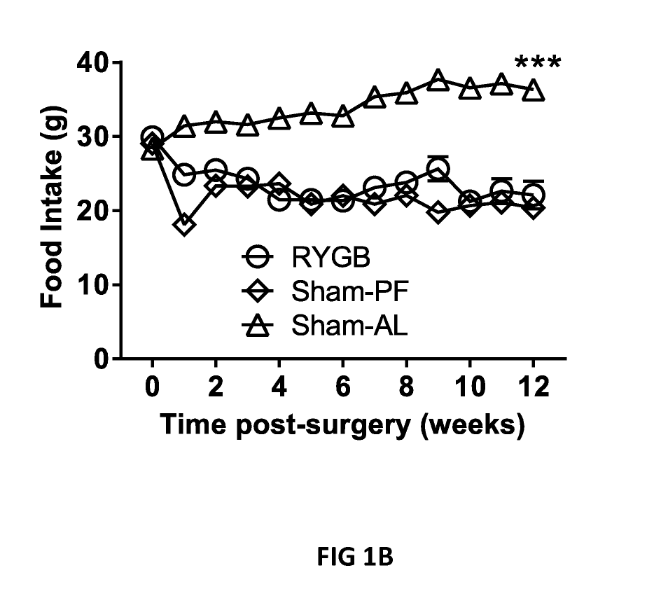

All data for beta cell function and immunohistochemistry are presented as a comparison between RYGB and Sham-PF, to quantify the changes in RYGB that were not due to reduced food intake, a common phenomenon associated with RYGB surgery due to reduced stomach size. Body and food intake data are presented for ZDF rats among the 3 groups (FIGS. 1A and B).

Surgical Procedure

RYGB surgery was performed under general anesthesia (1-3% isoflurane:oxygen provided to the inflow of the ventilator). Pre-surgical analgesia was provided by administering buprenorphine (0.05 mg/kg SC) and ceftriaxone (100 mg/kg, IM into the lateral thigh) was administered to prevent infection. To compensate for the fluid loss, lactated Ringer's solution (20-30 ml/kg, SC, twice) was given twice, prior to and immediately post-surgery. Animals were placed into the induction chamber with 4-5% isoflurane, and then intubated and connected to the ventilator. After attaining the full depth anesthesia a midline laparotomy was made freehand and the stomach was mobilized and divided by the stapling devices (ETS-Flex Ethicon Endo Surgery) to create a gastric pouch ˜30% of the original stomach size. Next, the jejunum was divided 15 cm below the ligament of Treitz and its distal end was sutured to the small opening in the anterior wall of the gastric pouch to create gastro jejunal end-to-side anastomosis. The proximal (duodenal) limb of the jejunum was anchored to the mid portion of the jejunum 10 cm below the gastro jejunal anastomosis in the end-to-side manner. Thus, RYGB arms were created with a biliopancreatic limb of 15 cm and aRoux limb of 10 cm. The abdomen was then closed, and 0.5 ml of 0.25% bupivacaine was infiltrated around the wound to reduce post-operative discomfort.

For Sham-operated rats, laparotomy was performed and the gut was periodically manipulated with cotton swabs without any surgery. Operative time was approximated to a similar degree as experienced by rats undergoing RYGB surgery.

All rats received buprenorphine (0.05 mg/kg SC) BID for 5 days post-operation. Lactate Ringer's solution (20-30 ml/kg) was given as needed and ceftriaxone (100 mg/kg) was administered once daily for the first 3 post-operative days. Rats did not have access to food and water for the first 24 hours after surgery, and then received liquid diet (Ensure Plus, vanilla flavor) ad libitum for the following 5 days, and returned to regular chow from day 7 post-surgery for the rest of the study until necropsy.

Body Weight and Food Intake

Body weight and food intake were measured daily for the first two weeks and then three times per week throughout the study for each cohort until their termination at weeks 3, 6 and 12 or 15 post-surgery. Pair-fed sham rats received the exactly same amounts of food consumed over 24 hours as their counterparts RYGB rats.

Oral Glucose Tolerance Test (OGTT) or Glucose Potentiation of Arginine-Induced Insulin Secretion (GPAIS)

OGTT or GPAIS tests were performed as a measurement of in vivo beta cell function at 3, 6, 9 and 12 or 15 weeks following surgery. Briefly, animals were fasted overnight (5:00 PM-8:00 AM) and on the test day body weight and blood glucose (BG) was measured (designated as 0 min timepoint). Next, rats were dosed either with oral glucose (2 g/kg dextrose) or IP with glucose+arginine (1 g/kg glucose, 600 mg/kg arginine; solution in PBS; administered at 4 ml/kg body weight). Blood glucose (determined with Embrace glucose meters) was measured from 0 to 180 min and plasma samples were collected for insulin analysis from 0 to 60 min. Food was returned after the 180 min time point.

Terminal Necropsy

Separate cohorts of rats underwent necropsy at week 3, week 6 or week 12 or 15 post-surgery for plasma and tissue collection. Animals from each cohort were divided into 2 subgroups for tissue collection for immunohistochemistry analysis and for gene expression profiling (GEP) via Affymetrix gene chip analysis followed by confirmation using Q-PCR.

Statistical Analysis

All data were analyzed using a Two-way ANOVA with post-hoc t-tests using Bonferroni's methods for differences between treatment and time points. Areas under the curve corrected for baseline for insulin (AUCB from 0-60 min for insulin) and basal fasting insulin (0 min time point) values between the three groups were analyzed by a One-way ANOVA with post-hoc t-tests using Bonferroni's methods. A p value of <0.05 or lower was taken to be statistically significant for RYGB-surgery group compared to Sham-PF rats. All data were analyzed and plotted using GraphPad Prism5 software (San Diego, Calif.).

Results

RYGB-surgery produced body weight loss compared to Sham-AL and Sham-PF rats (8-10%) at week 3, 6 and 12 in ZDF rats (FIG. 1A, **p<0.01, ***p<0.001 from Sham-AL). RYGB and Sham-PF groups were identical in food intake compared to Sham-Ad-lib rats (FIG. 1B, ***p<0.001, from Sham-AL) Results from GK rats were similar to that for ZDF rats (data not shown here).

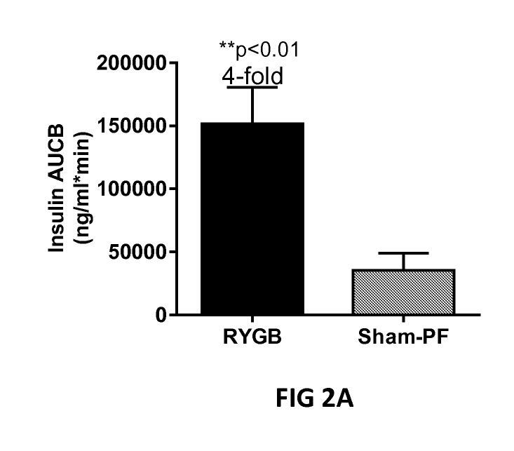

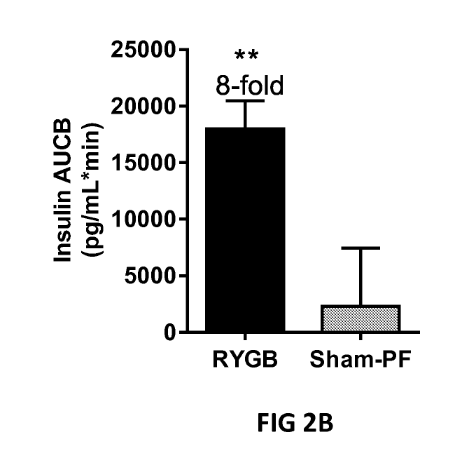

RYGB surgery produced significant increases (4- or 8-fold) in insulin secretion compared to Sham-PF (FIG. 2A depicts effects in ZDF rats and FIG. 2B shows effects in GK rats at week 12 post-surgery, **p<0.01) during an OGTT. These findings confirm the improvement in beta cell function post-RYGB, as reported previously. Our data strengthen the hypothesis that the effects are not due to caloric restriction per se, since the improvement in beta cell function by RYGB group was significantly better than shown by the Sham-PF rats.

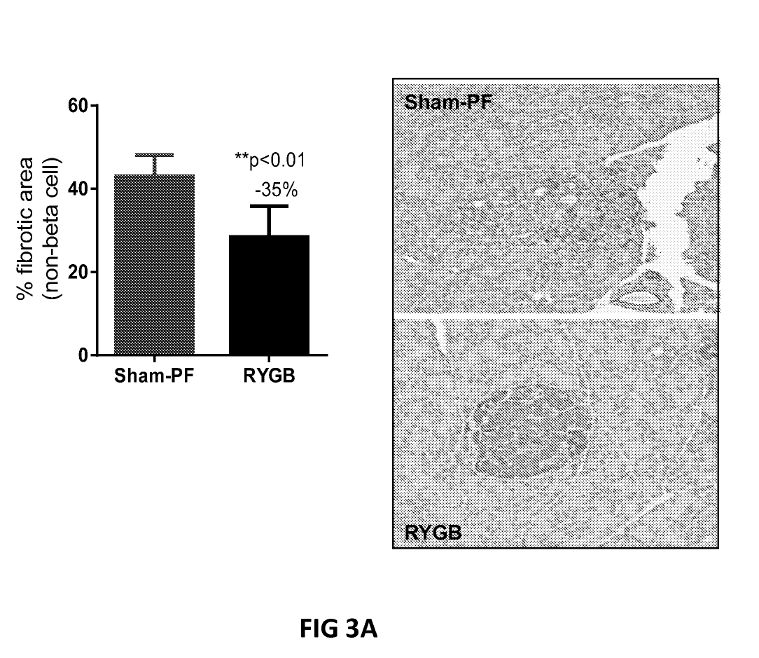

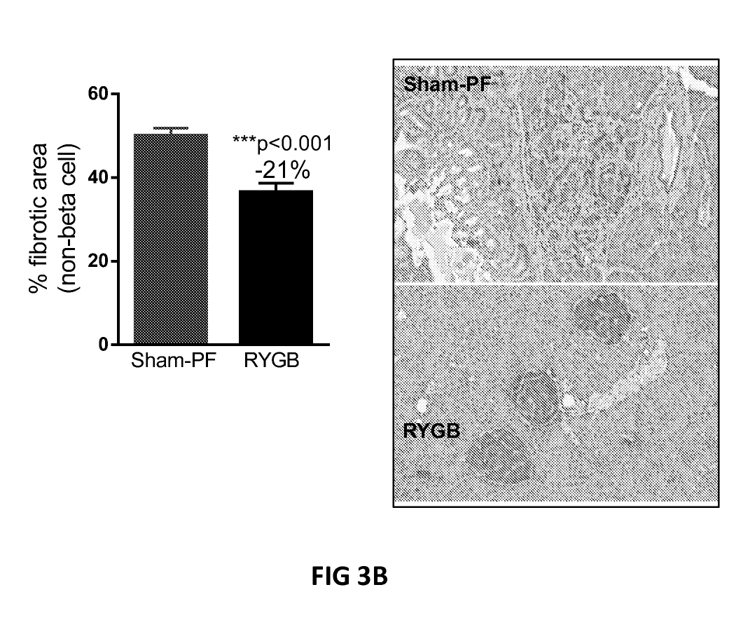

RYGB surgery produced significant decreases (21-35%) reduction in fibrotic area in islets from either ZDF (FIG. 3A) or GK (FIG. 3B) rats at weeks 12 and 15, respectively. Representative micrographs of the islets are shown in the right panels of FIGS. 3A and 3B. These findings are novel and indicate that RYGB surgery protected from worsening of islet health in these beta cell-impaired rats.

Table 1 shows changes in key plasma incretin hormones, such as glucagon-like peptide-1, GLP-1 and glucose-dependent insulinotropic polypeptide (GIP), that are known to regulate beta cell function. Following RYGB compared to Sham-PF at week 3, 6 and 12 post-surgery in ZDF rats, a significant increase in overnight fasted basal plasma GLP-1 (; total: 1.3-1.7-fold at weeks 3 and 6; active: 1.5-fold only at week 3) and GIP (;1.3-fold) levels were observed in RYGB rats compared to Sham-PF. Plasma GIP levels were significantly reduced (−33% from Sham-PF) at week 12 post-surgery. GK rats showed similar changes in GLP-1 and GIP levels (data not shown here)

TABLE 1
Plasma GLP-1 (active and total) and GIP (total)

| | Week 3 | | Week 6 | | Week 12 | |
|---|---|---|---|---|---|---|
| Plasma | RYGB | Sham-PF | RYGB | Sham-PF | RYGB | Sham-PF |
| GLP-1 total | 16.1 ± 1.73** | 9.7 ± 0.83 | 20.4 ± 2.7* | 15 ± 1.78 | 23.0 ± 1.13 | 26.5 ± 2.13 |
| (pg/mL) | | | | | | |
| GLP-1 active | 5.12 ± 0.36*** | 3.46 ± 0.27 | 5.1 ± 0.5 | 4.6 ± 0.38 | 6.7 ± 0.63 | 5.9 ± 0.59 |
| (pg/mL) | | | | | | |
| GIP total | 49.4 ± 5.55** | 37.4 ± 2.74 | 47.2 ± 3.24 | 47.5 ± 5.32 | 36.0 ± 2.89* | 54.6 ± 4.57 |
| (pg/mL) | | | | | | |

Consistent with the findings of elevated circulating GLP-1 levels in the clinic and in preclinical models following RYGB surgery, we observed that basal GLP-1 (both total and active) levels were increased (˜30%) by RYGB. The other incretin hormone GIP, while elevated in the beginning (at week 3) was decreased at 12 weeks post-surgery. Previous report in humans indicate both increase and reduction of GIP levels depending on the surgery and time elapsed (Rabiee A et al., (2011), J Surg Res 167: 199-205; Korner J et al., (2007), Sug Obes Relat Dis 3: 597-601). Taken together, these observations indicate that both upper and lower enteroendocrine cells responded to surgical manipulation in the gut, triggering changes in hormones in the gut-pancreas axis as reported before.

### Example 2

### Differential Expression of a Novel Gene Following RYGB Surgery

Gene expression profiling of the ileum of ZDF and GK rats after RYGB surgery showed a significant increased expression of a secreted protein; Seminal-vesicle antigen like-1 (Sval1) in the ileum. Follow-up with Q-PCR confirmed the increase in Sval1 gene expression. A phylogenic tree analysis showed that the human paralog of Sval1 gene is Prolactin-Inducible Protein (PIP). All in vivo efficacy studies shown on Example 3 were performed with both Sval1 as well as PIP, and the magnitude of the effects are similar between the two genes. The data shown here are with PIP only.

### Example 3

### In Vivo Efficacy Study with Hydrodynamic Injection of the Gene that Encodes PIP in Mice

To ascertain if this newly implicated factor can replicate some of the benefits shown by the RYGB surgery, a hydrodynamic gene delivery method was developed in-house. We tested the effect of delivery of DNA encoding the secreted factor PIP in vivo. The Fc-fusion proteins of PIP was encoded in pLEV113 vector, which was delivered via hydrodynamic injection (HDI) in high fat diet-induced obese (DIO) mice.

HDI Method:

Mice, that were maintained for 12 weeks on high fat diet (60% Kcal fat, Research Diets), were sorted into groups based on their body weights and fed blood glucose levels. On the day of HDI of the PIP vector or control pLEV113 vector (30 ug/mouse), mice were placed in a restrainer and tails were cleaned with 70% alcohol. A 27-G butterfly needle connected with a 3 ml syringe was inserted into the tail vain, with bevel facing up, and pulled backwards on the syringe plunger to ensure blood was drawn into the syringe. The desired volumes (7% of body weight, up to a total of 3.2 mL) of DNA construct solution was injected over a short period (5-10 seconds) by hand. The needle was then withdrawn and bleeding stopped by adding pressure to the injection site with gauze.

Functional Studies:

After the HDI procedure, mice were monitored twice daily in the first 48 hours and then once per day for a week. Basal fed blood glucose levels in mice were measured at day 14, and blood samples were collected to obtain plasma for measuring Fc level or for endogenous protein levels in the future.

At day 16 post-HDI an OGTT was conducted to evaluate glucose handling and beta cell function. Basal and glucose-stimulated glucose and insulin levels were measured over 2-3 hours during the OGTT.

At day 30, basal blood glucose levels, changes in weight or food intake, and another functional tests, such as an OGTT, were performed for a 2^{nd} time.

At day 40 post-HDI, the study was terminated by euthanization of mice and plasma collected for key metabolic biomarkers.

Results:

Initially, we found that HDI delivery of the Sval1-endocing vector improved beta cell function (40% increase in GSIS) in high fat DIO mice (data nor shown here). Next, we compared the in vivo efficacy of human PIP to mouse Sval1 via HDI administration in DIO mice, and found that the human PIP improved beta cell function similar to that of mouse Sval1.

In subsequent studies both human and mouse PIP (full length native and Fc-tagged) were tested via HDI delivery in high fat DIO mice

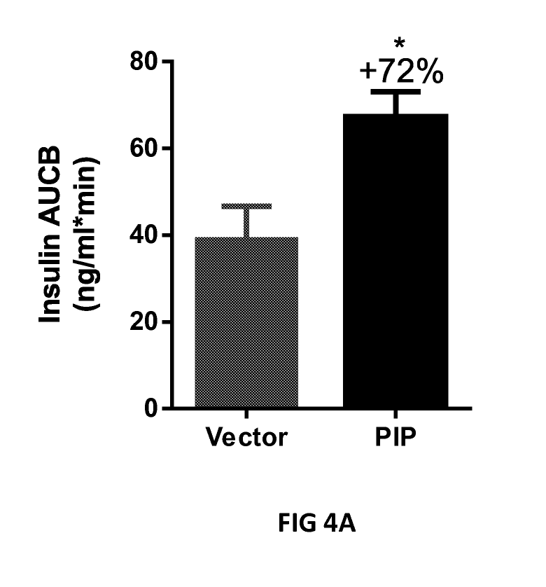

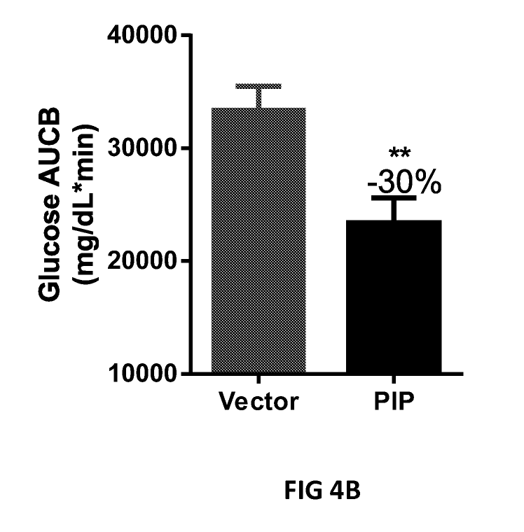

FIG. 4A shows representative data where mouse PIP increased GSIS, calculated by the insulin area under the curve (0-30 min) during an OGTT, by 72% (*p<0.05). PIP produced a 30% reduction (FIG. 4B; **p<0.01) in glucose area under the curve (0-120 min) during an OGTT in high fat DIO mice.

### Example 4

### In Vivo Efficacy Study with Recombinant PIP Fusion Protein in Mice

To ensure if the beneficial effects on GSIS and glucose loering is translable from HDI of vector encoding PIP gene and the recombinant PIP, studies were performed in DIO mice. Both Fc- and HSA-tagged fusion PIP proteins were used. An OGTT method was used for GSIS measuring pancreatic beta cell function in vivo.

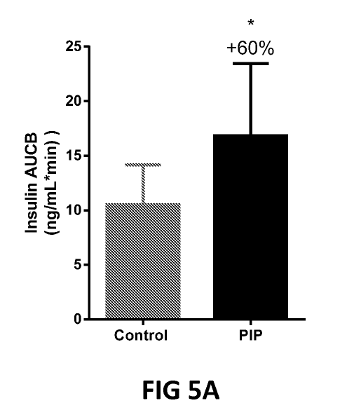

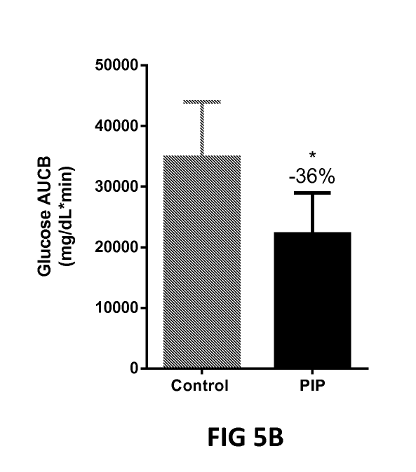

FIGS. 5A and B are graphical representations showing that a single intraperitoneal administration of a human serum albumin-recombinant human PIP fusion protein (at 0.3 mg/kg, IP) increased GSIS and improved oral glucose tolerance (OGT) in high fat DIO mice at 1 h post-dose. There was a 60% increase in insulin area under the curve (GSIS) (5A) and a 36% reduction in glucose area under the curve (5B) compared to the Vehicle control group; *p<0.05 by t-test.

### Example 5

### In Vivo Efficacy Study with Recombinant PIP Fusion Protein in Combination with Approved Therapeutics Exendin-4 and Metformin in Mice

To capture if recombinant PIP can also can be additive with currently approved therapeutics prescribed for T2DM, studies were performed co-administering PIP with either exendin-4 or metformin. An OGTT was given to quantitate glucose excursion in presence of exendin-4 in high fat DIO mice. An insulin tolerance test (ITT) was carried out to evaluate changes in insulin sensitivity in presence of metformin, an insulin sensitizer, in an obese insulin resistant diabetic ob/ob mice.

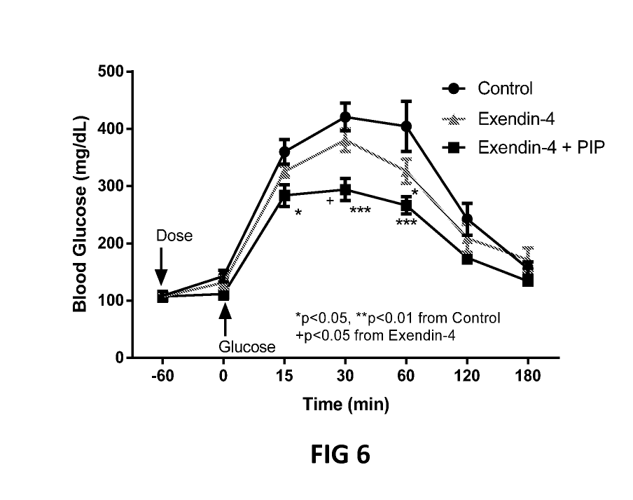

FIG. 6 is a graphical representation showing that a HSA-recombinant human PIP fusion protein (0.3 mg/kg, IP) was additive to Exendin-4 (0.3 ug/kg, IP) to improve oral glucose tolerance (OGT; dextrose 3 g/kg, oral given at 1 h post-dose) in high fat DIO mice. Data are presented as a time-course of glucose excursion *p<0.05, **p<0.01 from Control, +p<0.05 from Exendin-4 by a 2-way ANOVA with post-hoc analysis.

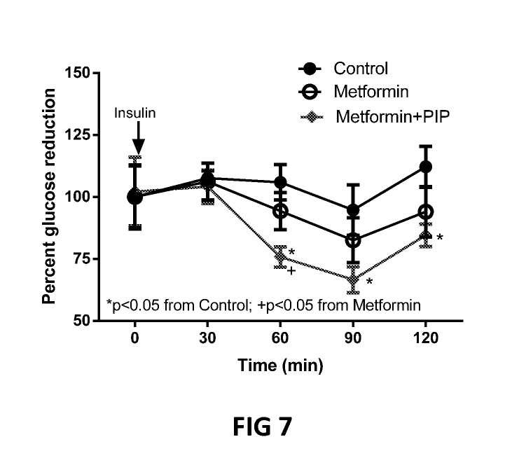

FIG. 7 is a graphical representation showing that the Fc-recombinant human PIP fusion protein (0.1 m/kg, IP) was additive to Metformin (100 mg/kg, oral) to improve insulin sensitivity as measured by an insulin tolerance test (ITT, Humulin 1.2 U/kg, IP was given at 1 h post-dose) in the insulin resistant ob/ob mice. Data are presented as a time-course of glucose excursion. *p<0.05 from Control, +p<0.05 from Metformin by a 2-way ANOVA with post-hoc analysis.

1. A method for treating a patient exhibiting one or more metabolic disorders, said method comprising administering to said patient a therapeutically effective amount of a PIP protein, PIP variant or PIP fusion protein.

2. The method of claim 1, wherein the metabolic disorder consists of one or more of the following: T2DM, pancreatic beta cell impairment, glucose intolerance, hyperglycemia, insulin resistance, obesity, dyslipidemia, NASH, metabolic syndrome, and other metabolic disorders.

3. The method of claim 2, wherein the metabolic disorder consists of T2DM.

4. The method of claim 1, said method comprising administering to said patient a pharmaceutical composition comprising a therapeutically effective amount of a PIP protein, PIP variant or PIP fusion protein, wherein said patient exhibits one or more metabolic disorders.

5. The method of claim 4, wherein the metabolic disorder consists of one or more of the following: T2DM, pancreatic beta cell impairment, glucose intolerance, hyperglycemia, insulin resistance, obesity, dyslipidemia, NASH, metabolic syndrome, and other metabolic disorders.

6. The method of claim 4, wherein the metabolic disorder consists of T2DM.

7. A method for reducing one or more of hyperglycemia, liver lipids, and weight gain in a patient in need, comprising administering to said patient a therapeutically effective amount of a PIP protein, PIP variant or PIP fusion protein.

8. A method for improving one or more of pancreatic beta cell function; insulin sensitivity; and glycemic control in a patient in need, comprising administering to said patient a therapeutically effective amount of a PIP protein, PIP variant or PIP fusion protein.
